(19) United States
(12) Patent Application Publication
(10) Pub. No.: US 2026/0000496 A1
(43) Pub. Date: Jan. 1, 2026

(54) MATERIALS AND METHODS FOR DRUG-INDUCED GINGIVAL OVERGROWTH

(71) Applicant: Optimed Technology, Inc., North Tonawanda, NY (US)

(72) Inventors: Praveen ARANY, Amherst, NY (US); Saeed Ur RAHMAN, Swabi (PK); Fatma ALKASSIMI, Buffalo, NY (US); Imran KHAN, Bethesda, MD (US); Ronald BROWN, Arlington, VA (US)

(21) Appl. No.: 19/320,719

(22) Filed: Sep. 5, 2025

Related U.S. Application Data
(62) Division of application No. 17/609,272, filed on Nov. 5, 2021.
(60) Provisional application No. 62/844,202, filed on May 7, 2019.

(51) Int. Cl. A61C 19/06; A61K 9/06; A61K 9/50; A61K 31/519; A61K 47/02; A61K 47/38

(52) U.S. Cl. CPC A61C 19/063; A61K 9/06; A61K 9/5031; A61K 9/5089; A61K 31/519; A61K 47/02; A61K 47/38

(57) ABSTRACT

Disclosed are gel compositions containing microspheres that encapsulate folic acid and optionally one or more drugs, one or more peptides, one or more growth factors, one or more nucleic acids, or the like, or combinations thereof. The compositions may be in or applied to a device, such as, for example, a dental prosthetic. The composition may be used to treat an individual afflicted with or suspected of having drug-induced gingival overgrowth (DIGO). The composition may be applied directly to the individual or applied through a device, such as, for example, a dental prosthetic. Also described is a method of method of making the composition, a method of making a device, and methods for using the composition.

### CROSS REFERENCE TO RELATED APPLICATIONS

This application is a divisional application of U.S. Patent Application of Ser. No. 17/609,272 filed on Nov. 5, 2021, which is a National Stage Application of International Application of PCT/US2020/031965 filed on May 7, 2020, which claims the benefit of priority to U.S. Provisional Application No. 62/844,202, filed on May 7, 2019, the disclosures of which are incorporated herein by reference.

### BACKGROUND OF THE DISCLOSURE

The status of the oral cavity (mouth) is important in reflecting the local and systemic health of the subject. The oral cavity has two part namely the vestibule and oral cavity proper that consists of hard and soft tissue structures. The soft tissue structures include the lips, mucosa, soft palate, floor of the mouth, tongue and gingiva. The gingiva is the mucosal tissue covering the cervical portion of the teeth and the alveolar process of the jaws. It is an external component of the periodontium that supports the tooth. Anatomically, the gingiva is divided into three main parts namely the attached gingiva, marginal gingiva and the interdental papilla. The healthy gingiva provides protection against mechanical injuries and microbial infections.

Histologically, the gingival consists of parakeratinized, stratified squamous epithelium and connective tissue. The epithelium plays a significant role in the immune defense responses against microorganisms and a variety of external injuries. The principle cell in the epithelium is the keratinocyte that secretes keratin and forms a protective physical barrier. On the other hand, the gingival connective tissue is primarily composed of fibroblasts, endothelial cells and immune cells that form its matrix, vessels and perform immune functions.

Clinically, the healthy gingiva is pale pink in color and described as “salmon” or “coral pink.” The normal gingiva has a rigid consistency and firmly attached to the teeth and underlining alveolar bone. Gingiva, like any other structure in the body, is susceptible to multiple pathological conditions that may alter its natural characteristic such as color, size, contour, shape, consistency or surface texture. A condition that leads to an alteration in the size and shape of the gingiva that often covers the clinical crown leading to esthetic and functional concerns is called gingival overgrowth.

Gingival overgrowths are categorized as inflammatory gingival overgrowth, hereditary gingival fibromatosis, systemic causes of gingival overgrowth and drug-induced gingival overgrowth (DIGO). The inflammatory gingival overgrowths are due to a pronounced inflammatory reaction initiated by local irritant such as plaque, calculus, microbial and trauma. Hereditary gingival fibromatosis (HGF), also known as an idiopathic hyperplasia, is a rare benign condition characterized by non-hemorrhagic, fibrous overgrowth of the attached gingiva. Systemic causes of gingival overgrowth may lead to localized or generalized gingival overgrowth. The most common systemic causes are pregnancy, hormonal imbalance, leukemia, and vitamins deficiency. Other causes of gingival overgrowth include lysosomal storage disorders, vascular disorders and the gingival overgrowths associated with teeth abnormalities. Gingival overgrowth associated with genetic defect can appear as a separate entity or as a part of a syndrome. It has been often associated with tooth eruption in the maxillary and mandibular second and third molars region. The bone is rarely involved, but the development of pseudo-pockets can lead to periodontal problems. Gingival overgrowth is commonly associated with different systemic diseases such as leukemia, Wegener's granulomatosis, Crohn's disease, Sarcoidosis and Tuberculous. Gingival overgrowth is also rarely associated with amylogenesis imperfecta, Hashimoto's thyroiditis, I-cell disease and multiple myeloma. The focus of this project is drug-induced gingival overgrowth that is elaborated further in the following sections.

Several well-known classes of drugs are known to induce DIGO and include three major categories namely, anti-convulsants (e.g., Phenytoin), immunomodulators (calcineurin inhibitors, e.g., Cyclosporine), and antihypertensives (calcium channel blockers eg; Nifedepine). Generally, these drugs show a high incidence of gingival overgrowth within treated patients with the highest incidence reported with anticonvulsants followed by immunosuppressant and antihypertensive drugs. The first report in 1939 described DIGO related to the anticonvulsants and antihypertensive with calcium channel blockers. DIGO was first reported as a side effect of cyclosporine (CsA) in 1972 and nifedipine in the early 1980s. The gingival overgrowth leads to several complications and contributes to an increased risk of dental decay, periodontal disease, occlusion problems and esthetic and speech disturbances. Seymour et al. in 1992 reported that 25-81% of CsA treated patients developed gingival overgrowth. Later, Kilpatrick et al. in 1997 showed that 97% of transplant recipient children who received CsA therapy developed gingival overgrowth. The prevalence of gingival overgrowth in association with cyclosporine and calcium channel blocker are 30% and 20%, respectively.

### SUMMARY OF THE DISCLOSURE

Disclosed herein are compositions (e.g., a gel comprising a plurality of microspheres encapsulating folic acid) suitable for treatment of DIGO and methods of making the compositions and using the compositions. Also provided are devices (e.g., dental devices, such as, for example, dental prosthetics) and methods of using the devices.

In an aspect, the present disclosure provides compositions comprising polymeric gel comprising a plurality of microspheres. The individual microspheres of the plurality of microspheres encapsulate folic acid.

In an aspect, the present disclosure provides methods for making a composition of the present disclosure.

In an aspect, the present disclosure provides devices. The devices may comprise a prosthesis and a composition of the present disclosure. The composition may be disposed in the prosthesis and/or on a surface of the prosthesis.

In an aspect, the present disclosure provides methods of making a device of the present disclosure.

In an aspect, the present disclosure provides a method for treating an individual afflicted with or suspected of having DIGO. The method may comprise utilizing a device of the present disclosure.

### BRIEF DESCRIPTION OF THE FIGURES

For a fuller understanding of the nature and objects of the disclosure, reference should be made to the following detailed description taken in conjunction with the accompanying figures.

FIG. 1 shows fibroblasts treated with TGF-β induce collagen expression.

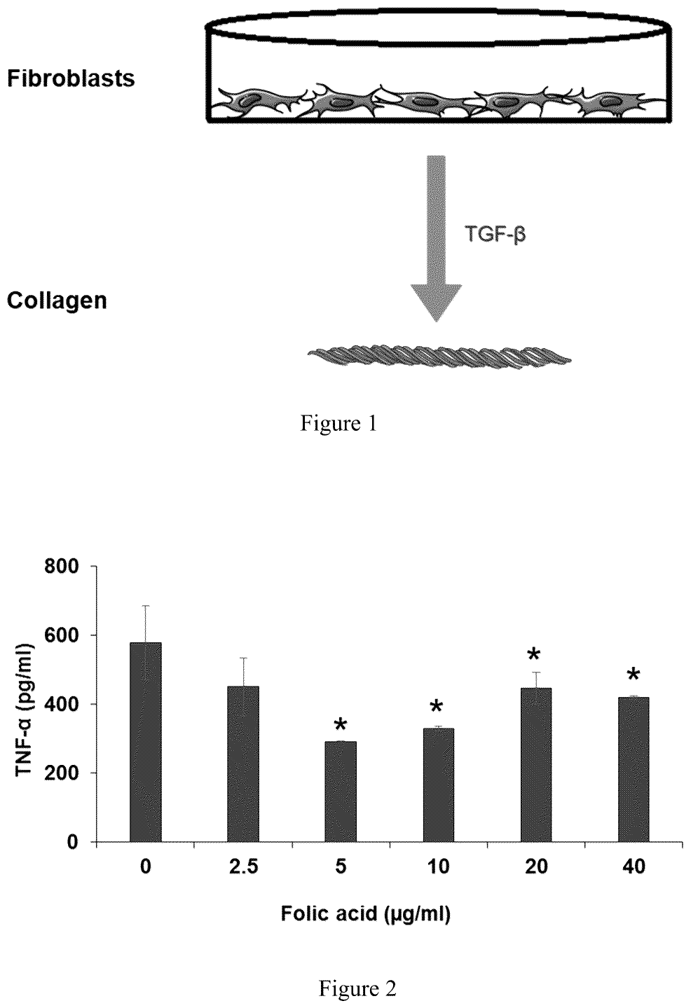

FIG. 2 shows folic acid reduces TNF-α oral keratinocytes. Oral Keratinocytes were seeded in 60 mm plates with a density of 2.5×10⁴/cm². After adhesion, the cells were treated with different concentration of folic acid (2.5 μg/ml-40 μg/ml) and amount of TNF-α was measured by ELISA (p<0.05), *significantly different from non-treated.

FIG. 3 shows folic acid reduces TNF-α in Oral Keratinocytes on LPS stimulation. Oral Keratinocytes were seeded in 60 mm plates with a density of 2.5×10⁴/cm². After adhesion, the cells were treated with LPS (10 μg/ml), CSA (10 μg/ml), and FA (10 μg/ml) for 24 hours. The amount of secreted TNF-α was determined by ELISA (p<0.05), *significantly different from non-treated, ^{#}significantly different from non-folic acid group.

FIG. 4 shows a schematic representation of the transfection factors bind to the Human TNF-α promoter and luciferase deletion constructs generated.

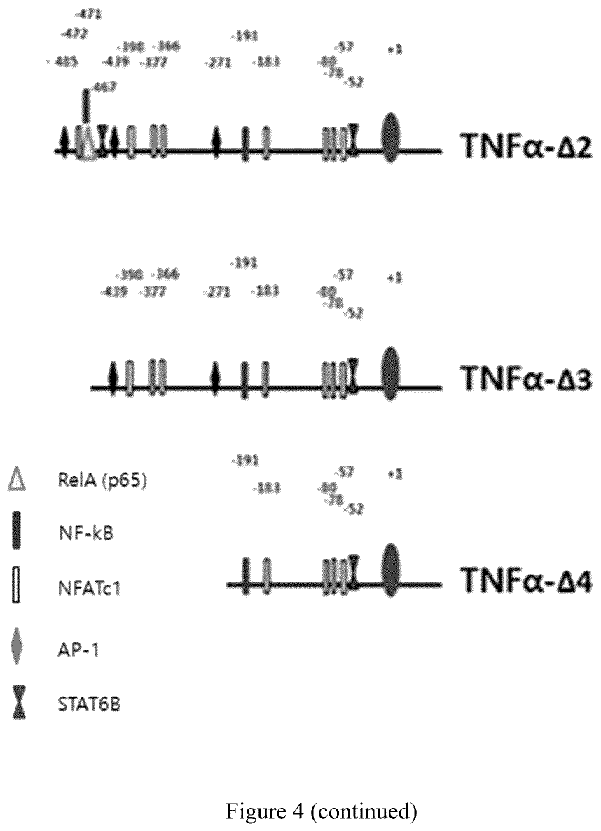

FIG. 5 shows folic acid inhibits promoter activity of TNF-α in Oral Keratinocytes cells. Oral Keratinocytes cells were transfected with Full length TNF-α promoter and four deletion constructs (Δ1−4) and treated with CSA (10 μg/ml) & LPS (10 μg/ml), and CSA (10 μg/ml), LPS (10 μg/ml) & Folic acid (10 μg/ml). TNF-α luciferase reporter activity were measured. (p<0.05), *significantly different from non-treated, ^{#}significantly different from non-folic acid group.

FIG. 6 shows folic acid inhibits CSA-induced expression of LITAF, p-P65, and MyD88 proteins in Oral Keratinocytes cells oral Keratinocytes were seeded in 60 mm plates with a density of 2.5×10⁴/cm² and treated with LPS (10 μg/ml), CSA (10 μg/ml), and FA (10 μg/ml) for 24 hours and determined the LITAF, MyD88, and p-p65 proteins level. significantly different from non-treated, ^{#}significantly different from non-folic acid group.

FIG. 7 shows folic acid inhibits CSA-induced activation of phospho-P65 in Oral Keratinocytes cells. Oral Keratinocytes cells were cultured, and treated with CSA, LPS or FA alone or with combinations and incubated for 24 hours. The immunofluorescent images were taken to determine the translocation of p-P65 to the nucleus. Cells were counterstained with Phalloidin (cytoskeleton) and DAPI (nucleus). P65 is green, DAPI is blue, and phalloidin is red.

FIG. 8 shows inhibition of MyD88 with different concentration of folic acid in Human Oral Keratinocytes cells (A) and inhibition of MyD88 at different time points after Folic acid treatment in Oral Keratinocytes cells (B). Oral Keratinocytes cells were cultured, and treated with FA with different concentration, and different time point (B) with a concentration of 10 μg/ml and determined the Dose-dependent effects (A) of FA on MyD88 assessed with western blots analysis at 24 hrs. Kinetics of MyD88 downregulation following FA treatments assessed with western blots (B).

FIG. 9 shows folic acid inhibits Phenytoin-induced TNF-α in Oral Keratinocytes cells. Oral Keratinocytes were seeded and treated with LPS (10 μg/ml), Phe (10 μg/ml), and FA (10 μg/ml) for 24 hours. The amount of secreted TNF-α was determined by ELISA. (p<0.05), *significantly different from non-treated, ^{#}significantly different from non-folic acid group.

FIG. 10 shows MyD88 expression is reduced by folic acid treatments. Oral Keratinocytes were seeded in 60 mm plates. The cells were treated with LPS (10 μg/ml), Phe (10 μg/ml), and FA (10 μg/ml) for 24 hours. The amount of MYD88 protein was determined by Western blot analysis (A). The protein bands were analyzed quantitatively (B) by using ImageJ software.

FIG. 11 shows LITAF expression is reduced by folic acid following drug treatments. Oral Keratinocytes were seeded in the plates. The cells were treated with LPS (10 μg/ml), Phe (10 μg/ml), and FA (10 μg/ml) for 24 hours. The amount of LITAF protein was determined by Western blot analysis (A). The protein bands were analyzed quantitatively (B) by using ImageJ software.

FIG. 12 shows dose dependent effect of two different kinds of LPS on LITAF expression and dose dependent effect of folic acid on LITAF expression. Oral Keratinocytes cells were treated with different serotypes (026:B6, and 055:B5) and concentrations (1 μg/ml & 10 μg/ml) of LPS (A), and different concentrations of folic acid (2.5-40 μg/ml) for 24 hours (C) and measured the amount of LITAF protein by Western blot analysis. The protein bands were analyzed quantitatively by using ImageJ software (B, D).

FIG. 13 shows folic acid reduces phosphorylation of NF-κB (p-P65). Oral Keratinocytes cell were seeded in the plates. The cells were treated with LPS (10 μg/ml), Phe (10 μg/ml), and FA (10 μg/ml) for 24 hours. The amount of phospho-p65 (p-P65) protein was determined by Western blot analysis (A). The protein bands were analyzed quantitatively (B) by using ImageJ software.

FIG. 14 shows folic acid reduced nuclear translocation of p-P65. Oral Keratinocytes cells were seeded in 24 well plates. The cells were treated with LPS (10 μg/ml), Phe (10 μg/ml), and FA (10 μg/ml) for 24 hours. Translocation of phospho-p65 (p-P65) protein to the nucleus was determined by fluorescence microscopy as shown in the green color. The morphology of cells and nucleus position were determined by palloidin and DAPI staining shown by red and blue, respectively.

FIG. 15 shows ChIP-qPCR assay demonstrates that LITAF binds to the TNF-α promoter. Oral Keratinocytes cells were seeded in 24 well plates. The cells were treated with LPS (10 μg/ml), Phe (10 μg/ml), LPS (10 μg/ml), and (FA (10 μg/ml) for 24 hours. CHIP-qPCR assay was performed to determine the binding of LITAF to the Human TNF-α promoter. *p<0.05 (treated vs non-treated), #p<0.05 (with vs without FA).

FIG. 16 shows folic acid treatments do not alter TLR2 and TLR4 expression. Oral Keratinocytes Cells were treated with only LPS (10 μg/ml) for 24 hours. The FACS assay analysis shows the amount of TLR4 receptor on the surface of Oral Keratinocytes cells (A). Cells were treated with LPS (10 μg/ml) and Phe (10 μg/ml) for 24 hours. The FACS assay analysis shows the amount of TLR2 and TLR4 receptor on the cells surface (B). Cells were treated with LPS (10 μg/ml), Phe (10 μg/ml), and FA (5, 10, and 10 μg/ml) for 24 hours. The FACS assay analysis shows the amount of TLR2 and TLR4 receptors on the surface of Oral Keratinocytes cells (C). FACS assay analysis shows the TLR2 and TLR4 receptors on the surface of Oral Keratinocytes cells after treatment with LPS, Phe, and different concentration of folic acid (D).

FIG. 17 shows folic acid treatments reduce TGF-β expression in Oral Fibroblast via inhibiting TNF-α signaling in Oral Keratinocytes. Human Oral Fibroblast (HOF) cells were cultured and treated with CSA, Phe, LPS, TGF-β inhibitor (SB), TNF-α inhibitor, rhTNF-α (TNF-α protein), and conditioned media (CM) collected from Oral Keratinocytes cells. ELISA results shows the amount of TGF-β secreted by HOF cells.

FIG. 18 shows folic acid reduces the collagen expression through TNF-α-activated TGF-β signaling in Human Oral Fibroblasts. Human Oral Fibroblasts (HOF) cells were seeded in 60 mm plates with a density of 2.5×10⁴/cm² and treated with condition (CM) media collected from Oral Keratinocytes containing LPS (10 μg/ml), CSA (10 μg/ml), Phenytoin (10 μg/ml), or Folic acid (10 μg/ml) and incubated for 24 hours. The Collagen type I alpha 1 (Col1a1) gene expression was determined by QPCR (A). HOF cells were treated with rTGF-β, SB-431542 (SB), rTNF-α, or TNF-α inhibitor (TNF-α inh). Quantitative real-time PCR results showing the expression of Col1a1 in HOF cells after the treatment (B). (p<0.05), *significantly different from non-treated, ^{#}significantly different from non-folic acid, TGFβ or TNF-α groups.

FIG. 19 shows (A) a schematic representation of paracrine signaling between Human Oral Keratinocytes and Human Oral Fibroblasts that is responsible for secreting TNFα that stimulates collagen production resulting in DIGO. (B) illustrates the signaling pathways that are disrupted by folic acid treatment resulting ain alleviation of DIGO.

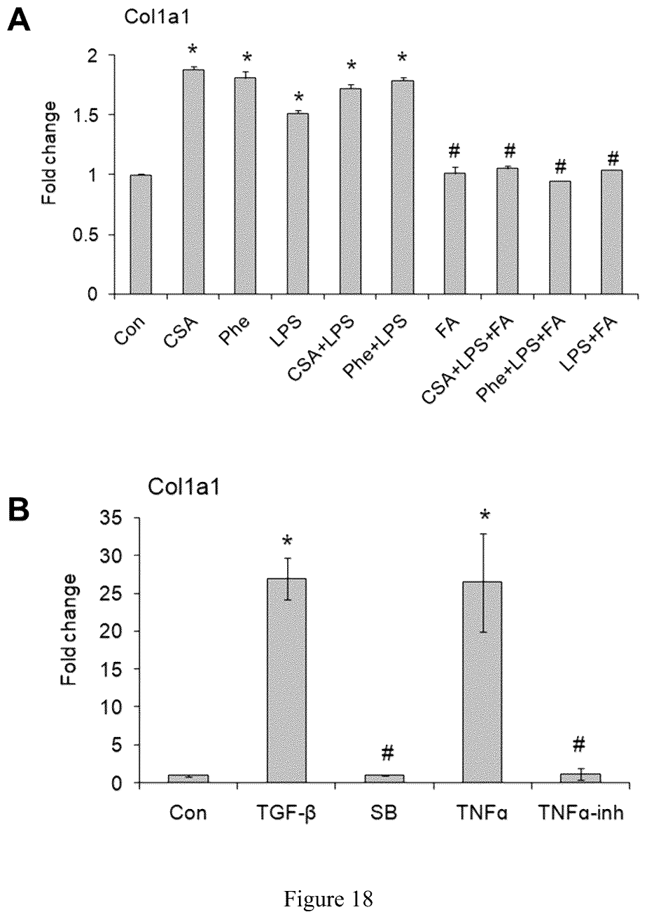

FIG. 20 shows sustained release of folic acid from polymeric microspheres.

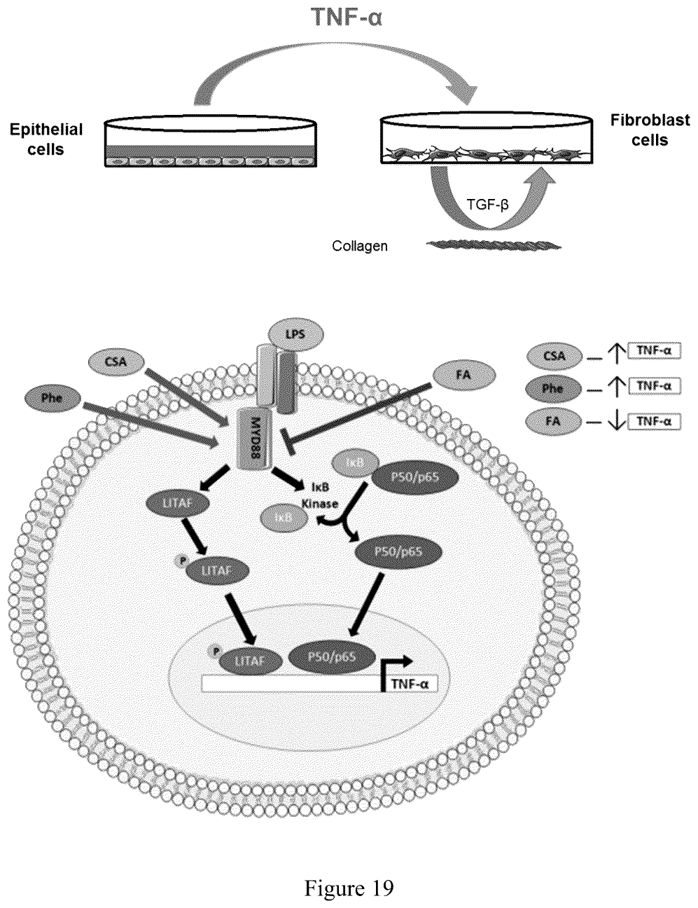

FIG. 21 shows animal study outlines for induction of DIGO and either prevention or treatment with folic acid.

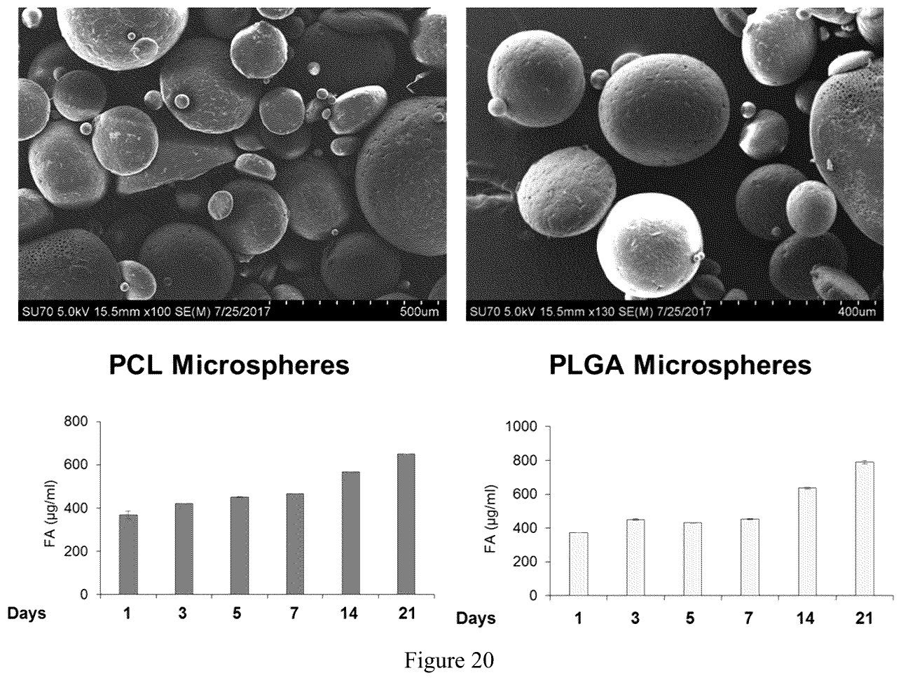

FIG. 22 shows an in vivo DIGO model to assess efficacy of folic acid treatments. SEM images of folic acid-encapsulated PLGA microspheres (left upper panel) mixed with methylcellulose gels (left lower panel) that was applied to mice gingiva (right lower panel). Silk sutures were placed around incisors to facilitate plaque accumulation and induce inflammation (right upper panel).

FIG. 23 shows in vivo DIGO models to assess efficacy of folic acid treatments. (A.) Folic acid treatment protocol in managing Drug-induced gingival overgrowth. Four weeks male C57/B6 mice were subjected to ligature placement and Cyclosporine (CsA) to induce gingival swelling, the top panel shows the time course of interventions while lower panels shows clinical images of placement and removal of sutures. (B.) The Folic acid (FA) treatment group was started at 1 week after establishment of DIGO and sutures were removed and CsA injections were stopped. The top panel shows the time line of interventions while lower panel shows clinical images of placement and removal or sutures and placement of FA gel.

FIG. 24 shows assessment of gingival swelling in treatment group. Following sacrifice, mice jaws were dissected and imaged using stereomicroscopy and specimens were photographed with a digital camera. In these studies, mice were divided into two groups where both groups received CsA and suture for 1 week followed by removal of sutures and stopping CsA injections. One group then received Folic acid applications daily (Treatment group) until the end of the study.

FIG. 25 shows assessment of gingival swelling in prevention group. Following sacrifice, mice jaws were dissected and imaged using stereomicroscopy. The specimens were photographed with a digital camera on their occlusal, buccal, and lingual surfaces. Mice were divided into three groups namely, untreated (control), Disease (CsA & sutures) and Prevention (CsA, sutures & Folic acid).

FIG. 26 shows images of the C57BL/6 mice gingiva in both groups over a time course in the prevention group and non-treated controls (A). Gingival samples were subjected to western blots to assess signaling pathways involved in DIGO (B).

FIG. 27 shows (A.) four weeks-male BALB/c mice had a suture place between the incisors and injected with Cyclosporine (CsA) alone or in combination with folic acid (CsA & FA) for prevention. Digital images were captured over a time course. (B.) Digital 3D planimetry was performed to assess gingival enlargements in two mice in both groups at Day 8.

FIG. 28 shows evaluation of gingival tissues in prevention group. Mice were sacrificed and total proteins isolated from dissected gingival tissues. Expression of LITAF, Phospho-p65, and MyD88 in gingival tissue lysates were examined by western blot analysis. Band intensity of western blot was normalized to β-actin and quantitated by densitometry. *P<0.05.

FIG. 29 shows folic acid reduced the expression of TNF-α in mice gingival tissues. Mice gingival tissue lysates were assessed for TNF-α levels using an ELISA (n=5, *p<0.05).

FIG. 30 shows validation of role of Myd88 in DIGO. DIGO was induced in male and female mice in wildtype and Myd88^{-/-} (knockout) mice and images of their gingival tissues were captured.

FIG. 31 shows wild type mice have significant DIGO induction compared to MyD88 knockout mice. Digital 3D planimetry to assess gingival enlargements in surrounding the incisors (A). Quantification of gingival enlargement in the two groups (B) (n=5*p<0.005).

FIG. 32 shows both genders MyD88 knockout mice have reduced DIGO. Digital 3D planimetry was performed to assess gingival enlargements in both groups of mice (A). Quantitative measurements of gingival enlargement in wild type and Myd88^{-/-} (knockout) mice (B).

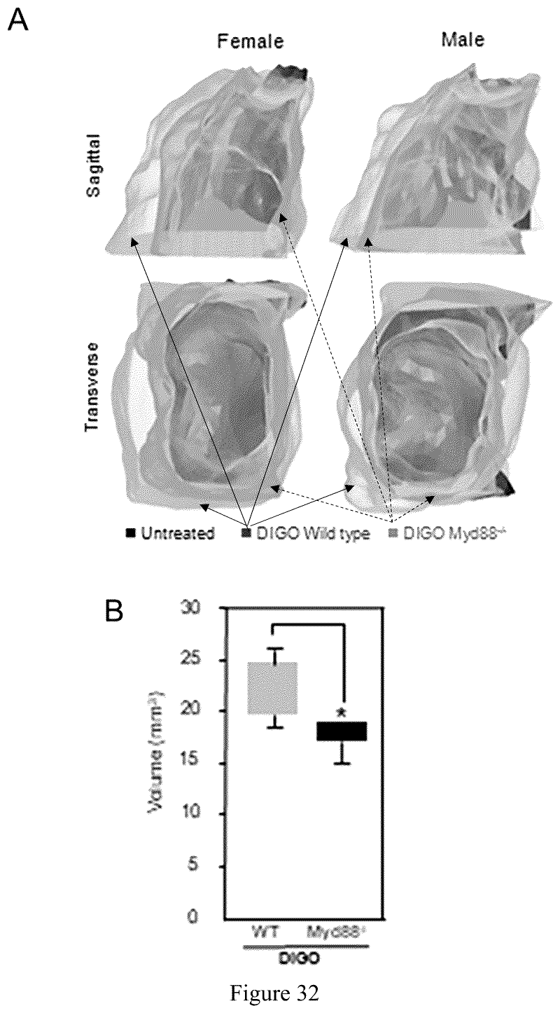

FIG. 33 shows amount of TNF-α in females gingival tissues of both groups of mice was performed (n=4/5 *p<0.05) (A). Amount of TNF-α in in males of both groups of mice was performed (n=3 *p<0.05) (B).

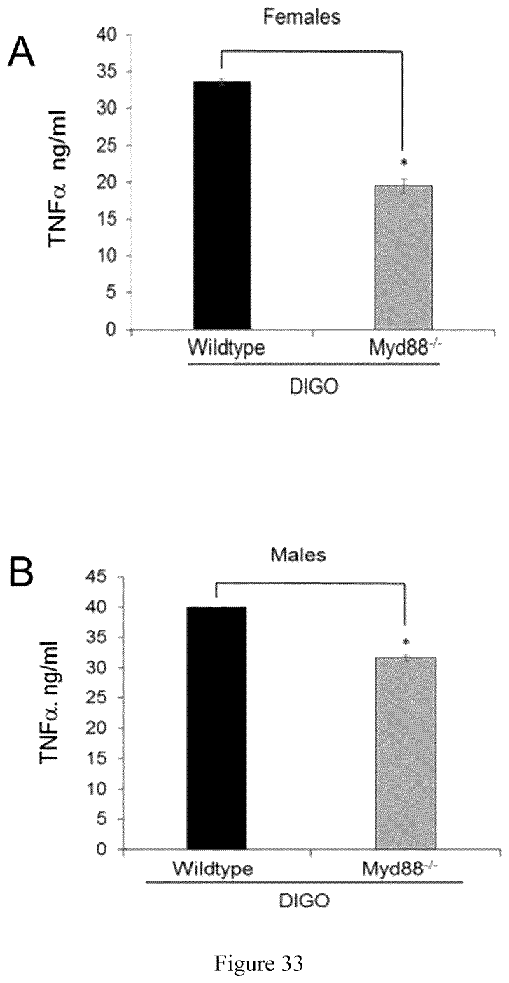

FIG. 34 shows a schematic illustration of the effect of TNF-α and TGF-β on collagen induction in human oral fibroblast (HOF) cells.

FIG. 35 shows western blot analysis that shows the ability of folic acid on downregulation of MyD88 (A), LITAF (B), and NF-κB (C) in normal oral keratinocyte cells.

FIG. 36 shows a controlled release system for folic acid treatments. Folic acid was encapsulated in polylactic-colycolide (PLG) microspheres that were visualized using scanning electron microscopy (SEM) (A). These microspheres were embedded within a methylcellulose gel to enable oral applications and visualized using SEM (B). Controlled release of folic acid of a protracted time course was examined using spectroscopy and cumulative release was calculated (C).

FIG. 37 shows folic acid treatment in managing DIGO. In a pilot study, mice were used to develop an in vivo model of DIGO by placing sutures and cyclosporin (CsA) i.p. injections. To examine efficacy of folic acid (FA) as a treatment, supplementation of FA in drinking water and daily applications of an FA gel was performed.

FIG. 38 shows clinical images of mice study. (A) Four week male BALB/c mice had a suture placed between the incisors and injected with CsA alone or in combination with folic acid (CsA+FA) and digital images were captured over a time course. (B) Higher power images of gingival tissues in two mice in both groups at day 8.

FIG. 39 shows a pilot study in BALB/c mice. In the first study, animals had sutures placed around their incisors and injected intraperitoneally with CsA to induce DIGO. Another group was concurrently treated with folic acid (FA) gel in order to prevent disease. (A) Stereomicroscopic images of gingival swelling in both groups in three mice are shown. The right panels show inadvertent loss of sutures in some mice in both groups but continued CsA injections. (B) Digital imaging and 3D volumetric reconstruction was performed at 8 days to evaluate gingival swelling. (C) Gingival tissues were excised and stained for Myd88 expression, dotted line denotes the epithelial basement membrane.

FIG. 40 shows clinical images in mice experiment 2 in C57/B6 mice. (A) Images show suture placement in gingiva around the two lower central incisors in 4 week male mice. (B) Clinical images of CsA and CsA with FA group over a time course.

FIG. 41 shows 3D morphometric analyses for gingival overgrowth. Digital images of gingival mice tissues from control (grey, no interventions), disease (red, sutures and CsA), and preventive treatment (blue, suture, CsA and folic acid). Using a representative sample, incisor teeth were cropped out digitally and images were overlaid to highlight the changes in gingival swelling noted at day 14 between (A) control and disease, (B) control and treated, (C) disease and treated, and (D) all three conditions. 3D volumetric analyses were performed in the disease and treatment, n=5 and statistical significance is noted by * where p<0.005.

FIG. 42 shows immunostaining analyses of gingival tissues. Mice were sacrificed and gingival tissue was processed for immunostaining. Three markers mediating gingival overgrowth, namely Myd88, LITAF, and NF-κB expression was assessed in both disease and folic acid preventative (A) and treatment (B) groups and compared to control (untreated) mice.

FIG. 43 shows tissue analyses for molecular markers. Following sacrifice, mice gingival tissue was routinely processed for immunoblotting and expression of Myd88 (A), LITAF (B), and phospho p65 (C) were examined. Tissues from three mice are shown here and densitometry quantitation of the bands for normalized expression are denoted below.

FIG. 44 shows clinical images in transgenic mice. Sutures were placed in the gingiva between incisors of female (A) and male mice (B) along with CsA injections (i.p.) daily. Clinical images were collected with a digital camera over the time course.

FIG. 45 shows gingival tissue analyses by 3D morphometry. Will type Myd88^{-/-} (knockout) mice were subjected to suture placements and daily injections of CsA for 2 weeks and then sacrificed. Gingival morphology was examined using 3D digital morphometry and representative volumes were reconstructed and quantitated in female (A & B, n=4/5) and male (C & D, n=3) mice. Statistical significance is denoted as * where p<0.05.

FIG. 46 shows histological analyses of human samples. Formalin fixed paraffin embedded sections were stained with hematoxylin and eosin. Microscopic evaluation was performed on these sections and demonstrated discrete features in various areas (A-F) likely representing the spectrum of the etiopathological process.

FIG. 47 shows immunostaining analyses of gingival tissues. Following IRB approval, human gingival tissues from patients with DIGO were subjected to immunostaining for Myd88 expression and imaged by using a light microscope and digital camera.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

Disclosed herein are compositions (e.g., a gel comprising a plurality of microspheres encapsulating folic acid) suitable for treatment of DIGO and methods of making the compositions and using the compositions. Also provided are devices (e.g., dental devices, such as, for example, dental prosthetics) and methods of using the devices.

As used in this disclosure, the singular forms “a”, “an”, and “the” include plural references and vice versa, unless the context clearly dictates otherwise. Thus, for example, references to “the method” includes one or more methods, and/or steps of the type described herein.

The terms “oral cavity” and “oral” as used herein refers to all of the anatomical structures that make up the mouth and oropharynx of an individual such as but not limited to the lips, cheeks, tongue, hyoid bone, teeth, gums, jaw bone (mandible), alveolar bone, salivary glands, tonsils, adenoids, hard and soft palate, uvula, tempomandibular joint, epiglottis, and all connective and epithelial tissues.

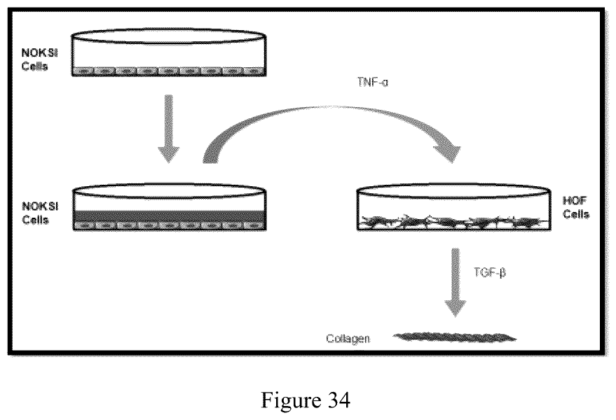

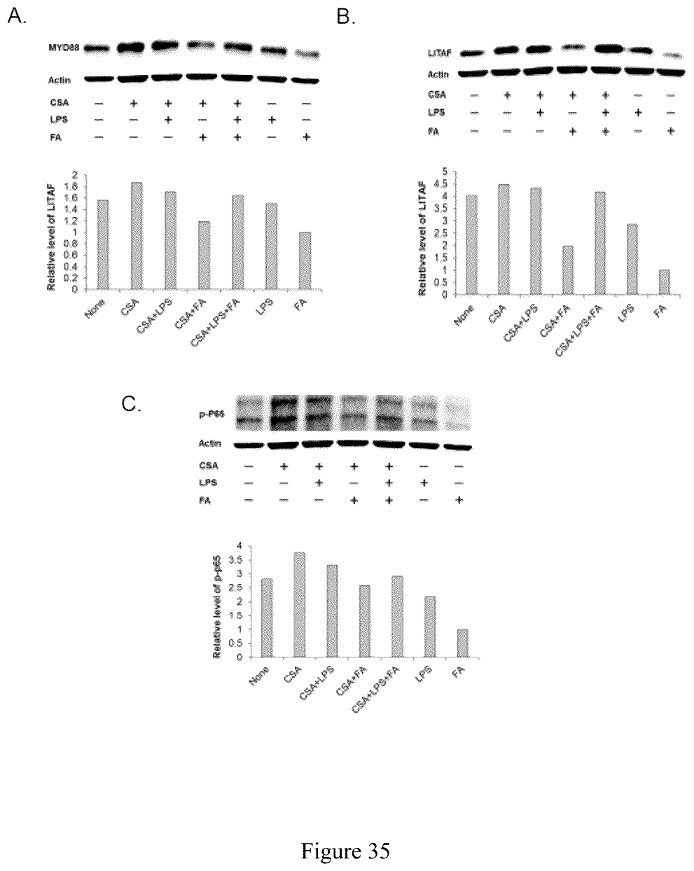

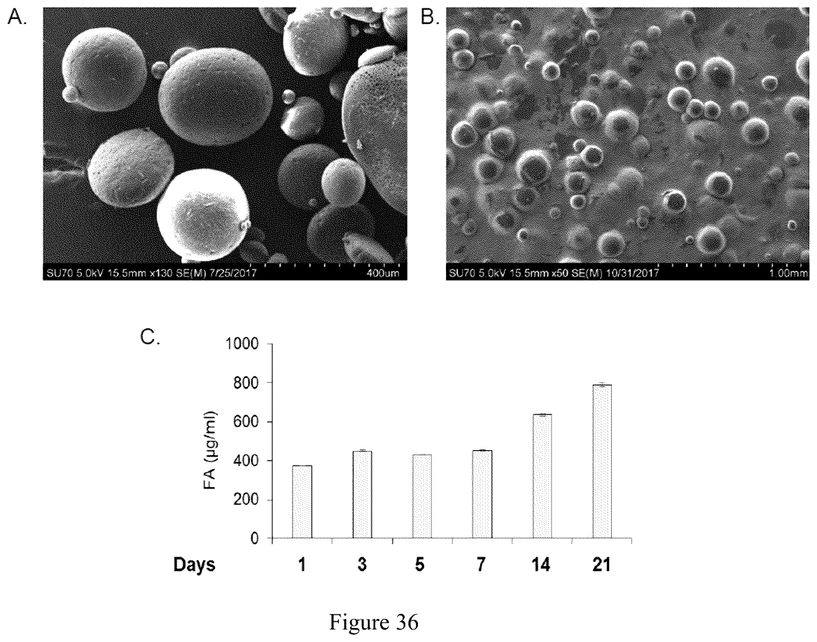

For the example, the present disclosure describes examination of the etiopathogenesis of DIGO in vitro, human oral fibroblasts and keratinocytes were treated with Cyclosporine (CsA) and Lipopolysaccharide (LPS) to simulate the disease (FIG. 34). Both treatments were noted to robustly induce MyD88 expression in human oral keratinocytes (FIG. 35). In order to deliver the Folic acid treatment, a controlled release system with polylactic glycolic acid (PLGA) and methylcellulose gel were used (FIG. 36). Release kinetics of folic acid increased over time and noted to continue until day 21. The amount of the released Folic acid was approximate 400 μg/ml on day 1 and reached 800 μg/ml on day 21 due to bulk deterioration of polymer.

In an aspect, the present disclosure provides compositions comprising polymeric gel comprising a plurality of microspheres. The individual microspheres of the plurality of microspheres encapsulate folic acid.

Various polymeric gels may be used. The polymeric gels may be formed from an aqueous solution comprising a gellating compound. Various gellating compounds may be used. Examples of gellating compounds include, but are not limited to, methylcellulose, chitosan, starch, polylactic glycolic acid, and the like, and combinations thereof. In various examples, the polymeric gel comprises methylcellulose (e.g., 5% methylcellulose) and phosphate buffered saline (PBS). The aqueous solution may be water or a buffered water solution.

The microspheres may be formed from (e.g., comprise) various materials (e.g., microsphere precursors). Non-limiting examples of microsphere precursors include polylactic glycolic acid, polycaprolactone (PCL), chitosan, starch, polylactic acid, alginate, and the like, and combinations thereof. In various examples, the microspheres are polylactic glycolic acid microspheres, polycaprolactone (PCL) microspheres, chitosan microspheres, starch microspheres, polylactic acid microspheres, poly (ester amide) microspheres, alginate microspheres, and the like, and combinations thereof. In various examples, the microspheres are polylactic glycolic acid microspheres. The microspheres are biocompatible and biodegradable. The microspheres may be suitable for sustained and controlled release of folic acid in the vicinity of a DIGO infection. The concentration of microspheres in the composition may be 0.001 to 0.07 mg/μL, including every 0.01 mg/μL value and range therebetween. In various examples, the microsphere concentration is about 0.06 mg/μL. The microspheres may comprise PVA.

The individual microspheres of the plurality of microspheres encapsulate folic acid. The concentration of folic acid in the composition may be 2.5 to 150 μg/mL, including all 0.01 μg/mL values and ranges therebetween (e.g., 2.5 to 40 μg/mL). In various examples, the concentration of folic acid is 100 μg/mL.

In various examples, the individual microspheres of the plurality of microspheres further comprise (e.g., encapsulate) one or more drugs, one or more peptides, one or more growth factors, one or more nucleic acids, and the like, and combinations thereof. The one or more drugs may be the same or different (e.g., structurally the same or different). Non-limiting examples of drugs include antibiotics, anti-inflammatories (e.g., NSAIDs, opioids, and the like, and combinations thereof), antibodies, and the like, and combinations thereof.

Examples of antibiotics include, but are not limited to, penicillins (e.g., Flucloxacillin (Flopen, Flucil), Amoxicillin+clavulanate (Augmentin, Clamoxym), Piperacillin+tazabactam (Tazocin)), cephalosporins (e.g., Cephalexin (Keflex, Ibilex), Cephazolin (Kefzol), Ceftriaxone (Rocephin)), macrolides (e.g., Azithromycin (Zithromax), Roxithromycin (Rulide)), azoles (e.g., Fluconazole (Diflucan), Voriconazole (Vfend)), and guanine analogues (e.g., Aciclovir (Zovirax), Valaciclovir (Valtrex)).

Non-limiting factors of growth factors include PDGF, EGF, TGF-α, TGF-β, KGF, FGF, IL-1, IGF-1, VEGF, SDF, BMPs, Wnts, IGF, IL-2, IL-3, IL-4, IL-5, IL-6, IL-7, LIF, IGF-2, MSP, MSF, TGF-α, Ang, AM, GDF-8, NGF, SHH, NGF, PDGF, FGFR, TNF, and the like, and combinations thereof.

Non-limiting examples of antibodies include anti-BMP, anti-TGF-β, anti-TNF-α, and the like, and combinations thereof.

In an aspect, the present disclosure provides methods for making a composition of the present disclosure.

A method of making a composition of the present disclosure may comprise (a) fabricating a plurality of microspheres, wherein the individual microspheres encapsulate folic acid; and (b) mixing the plurality of microspheres with an aqueous solution comprising a gellating compound. In various examples, the gellating compound is methylcellulose.

In various examples, fabricating the plurality of microspheres comprises a double emulsion solvent evaporation method. The double emulsion solvent evaporation method may comprise (a) mixing an amount of folic acid with a solution of a microsphere precursor: (b) adding a solution of polyvinyl acetate (PVA) to the microsphere precursor solution; (c) mixing microsphere precursor and PVA solution; (d) centrifugating the microsphere precursor and PVA solution such that a pellet and supernatant is formed; (e) collecting the supernatant; and (f) concentrating (e.g., lyophilizing) the supernatant such that a solid is formed, where the solid is the plurality of microspheres. More specifically, a 100-μL quantity of folic acid dissolved in molecular-grade water may be pipetted into 1 mL of 5% PLG in ethyl acetate and sonicated on 50% magnitude for 1 min. A second emulsion comprising 1% polyvinyl acetate (PVA) and 7% ethyl acetate may be added and vortexed for 15 sec. The solution may then be added to a 1% PVA solution with continuous stirring for 3 hrs at room temperature and filtered through a 0.2-μm filter, collected by centrifugation at 2,000 rpm for 10 min, freeze-dried for 16 hrs, and stored at −20° C.

In an aspect, the present disclosure provides devices. The devices may comprise a prosthesis and a composition of the present disclosure. The composition may be disposed in the prosthesis and/or on a surface of the prosthesis.

The device may be a dental prosthetic (e.g., a 3D-printed polymer dental prosthetic). The dental prostheses may be suitable to rebuild previously existing dental prostheses or for new prostheses. Examples of dental prostheses include, but are not limited to, splints, mouth guards, full dentures, partial dentures, fillings, and the like. The device may be made from (e.g., comprise) polymethylmethacrylate (PMMA), bis-GMA, polyester, polyacrylate, polyvinyl, epoxy, or the like, or a combination thereof.

In an aspect, the present disclosure provides methods of making a device of the present disclosure.

A method of making a device (e.g., a dental prosthesis, such as, for example, a splint, mouth guard, full denture, partial denture, or filling, or the like) may comprise: (a) obtaining a scan, mold, or model of an individual's oral cavity where the device will fit; (b) fabricating a device corresponding to the individual's scan, mold, or model of the oral cavity with a polymer (e.g., a PMMA polymer); (c) generating the composition of the present disclosure; and (d) coating device with the composition.

In an aspect, the present disclosure provides a method for treating an individual afflicted with or suspected of having DIGO. The method may comprise utilizing a device of the present disclosure.

A method of treating an individual may comprise administering to the individual a composition of the present disclosure for a sufficient time to treat the DIGO. In various examples, the composition may be directly applied to the afflicted area.

A method of treating an individual may comprise: (a) obtaining a scan, mold, or model of the individual's oral cavity where a device (e.g., a dental prosthesis, such as, for example, a splint, mouth guard, full denture, partial denture, or filling, or the like) will fit; (b) fabricating the device corresponding to the individual's scan, mold, or model of the oral cavity with a polymer (e.g., a PMMA polymer); (c) generating the composition of the present disclosure; (d) coating the device with the composition; (e) administering the completed device to the individual for sufficient time to treat the DIGO; and (f) optionally, monitoring the individual's response to the device treatment.

In various examples, the polymer is chosen from polymethylmethacrylate (PMMA) polymers, bis-GMA polymers, polyester polymers, polyacrylate polymers, polyvinyl polymers, epoxy polymers, and the like, and combinations thereof.

The steps of the method described in the various embodiments and examples disclosed herein are sufficient to carry out the methods of the present invention. Thus, in an embodiment, the method consists essentially of a combination of the steps of the methods disclosed herein. In another embodiment, the method consists of such steps. The following Statements describe various examples of the present disclosure.
Statement 1. A composition comprising a polymeric gel comprising a plurality of microspheres, where the individual microspheres of the plurality of microspheres encapsulate folic acid.
Statement 2. A composition according to Statement 1, where the polymeric gel is chosen from a methylcellulose gel, chitosan gel, polylactic glycolic acid gel, and combinations thereof.
Statement 3. A composition according to Statement 2 or Statement 2, where the polymeric gel is methylcellulose gel.
Statement 4. A composition according to any one of the preceding Statements, where the polymer gel comprises phosphate buffered saline and 1-5% weight by volume methylcellulose, including every 0.1% by weight value and range therebetween (e.g., 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5).
Statement 5. A composition according to Statement 4, where the methylcellulose concentration is 3% weight by volume.
Statement 6. A composition according to any one of the preceding Statements, where the plurality of microspheres are chosen from polylactic glycolic acid microspheres, polycaprolactone (PCL) microspheres, chitosan microspheres, starch microspheres, polylactic acid microspheres, poly (ester amide) microspheres, alginate microspheres, and the like, and combinations thereof.
Statement 7. A composition according to any one of the preceding Statements, where the microspheres are polylactic glycolic acid microspheres.
Statement 8. A composition according to any one of the preceding Statements, where the concentration of folic acid is 2.5 to 150 μg/mL, including every 0.01 μg/mL value and range therebetween (e.g., 2.5 to 40 μg/mL).
Statement 9. A composition according to any one of the preceding Statements, where the concentration of folic acid is 100 μg/mL.
Statement 10. A composition according to any one of the preceding Statements, where the concentration of microspheres is 0.001 to 0.07 mg/μL, including every 0.001 mg/μL value and range therebetween.
Statement 11. A composition according to any one of the preceding Statements, where the concentration of microspheres is 0.06 mg/μL.
Statement 12. A composition according to any one of the preceding Statements, where the plurality of microsphere(s) further comprise one or more drugs, one or more peptides, one or more growth factors, one or more nucleic acids, or the like, or combinations thereof.
Statement 13. A composition according to Statement 12, where the one or more drugs are chosen from antibiotics, anti-inflammatoires, antibodies and combinations thereof. The one or more drugs may be the same or different (e.g., structurally the same or different). Non-limiting examples of drugs include antibiotics, anti-inflammatoires (e.g., NSAIDs, opioids, and the like, and combinations thereof), antibodies, and the like, and combinations thereof. Examples of antibiotics include, but are not limited to, penicillins (e.g., Flucloxacillin (Flopen, Flucil), Amoxicillin+clavulanate (Augmentin, Clamoxym), Piperacillin+tazabactam (Tazocin)), cephalosporins (e.g., Cephalexin (Keflex, Ibilex), Cephazolin (Kefzol), Ceftriaxone (Rocephin)), macrolides (e.g., Azithromycin (Zithromax), Roxithromycin (Rulide)), azoles (e.g., Fluconazole (Diflucan), Voriconazole (Vfend)), and guanine analogues (e.g., Aciclovir (Zovirax), Valaciclovir (Valtrex)). Non-limiting factors of growth factors include PDGF, EGF, TGF-α, TGF-β, KGF, FGF, IL-1, IGF-1, VEGF, SDF, BMPs, Wnts, IGF, IL-2, IL-3, IL-4, IL-5, IL-6, IL-7, LIF, IGF-2, MSP, MSF, TGF-α, Ang, AM, GDF-8, NGF, SHH, NGF, PDGF, FGFR, TNF, and the like, and combinations thereof. Non-limiting examples of antibodies include anti-BMP, anti-TGF-β, anti-TNF-α, and the like, and combinations thereof.
Statement 14. A method for making the composition according to any one of the preceding Statements, comprising: (a) fabricating a plurality of microspheres, wherein the individual microspheres encapsulate folic acid; and (b) mixing the plurality of microspheres with an aqueous solution comprising a gellating compound, where the composition of according to any one of the preceding Statements is formed.
Statement 15. A method according to Statement 14, where the gellating compound is chosen from methylcellulose, chitosan, starch, polylactic glycolic acid, and the like, and combinations thereof.
Statement 16. A method according to Statement 14 and Statement 15, where the gellating compound is methylcellulose.
Statement 17. A method according to any one of Statements 14-16, where the fabricating comprises a double emulsion solvent evaporation method.
Statement 18. A method according to Statement 17, where the double emulsion solvent evaporation method comprises (a) mixing an amount of folic acid with a solution of a microsphere precursor: (b) adding a solution of polyvinyl acetate (PVA) to the microsphere precursor solution; (c) mixing microsphere precursor and PVA solution; (d) centrifugating the microsphere precursor and PVA solution such that a pellet and supernatant is formed; (e) collecting the supernatant; and (f) concentrating the supernatant such that a solid is formed, where the solid is the plurality of microspheres.
Statement 19. A method according to Statement 18, where the double emulsion solvent evaporation method further comprises filtering.
Statement 20. A method according to Statement 18 or Statement 19, where the concentrating is lyophilizing.
Statement 21. A device, comprising: (a) a prosthesis; and (b) a composition according to any one of Statements 1-13, where the composition is disposed in the prosthesis and/or on a surface of the prosthesis.
Statement 22. A device according to Statement 21, where the prosthesis is chosen from a splint, mouth guard, full denture, partial denture, and filling.
Statement 23. A device according to Statement 21 or Statement 22, where the polymeric gel is a methylcellulose gel.
Statement 24. A device according to any one of Statements 21-23, where the plurality of microspheres are polylactic glycolic acid microspheres.
Statement 25. A device according to any one of Statements 21-24, where the plurality of microsphere(s) further comprise one or more drugs, one or more peptides, one or more growth factors, one or more nucleic acids, or the like, or combinations thereof.
Statement 26. A device according to any one of Statements 21-25, where the prosthesis comprises polymethylmethacrylate (PMMA), bis-GMA, polyester, polyacrylate, polyvinyl, epoxy, or a combination thereof.
Statement 27. A device according to any one of Statements 21-26, where the device is 3D printed.
Statement 28. A method of making a device comprising: (a) obtaining a scan, mold, or model of an individual's oral cavity where the device will fit; (b) fabricating a device corresponding to the individual's scan, mold, or model of the oral cavity with a polymer; (c) generating the composition according to any one of Statements 1-13; and (d) coating device with the composition.
Statement 29. A method according to Statement 28, where the device is a dental prosthesis.
Statement 30. A method according to Statement 29, where the dental prosthesis is chosen from a splint, mouth guard, full denture, partial denture, and filling.
Statement 31. A method according to any one of Statements 28-30, where the polymer is chosen from polymethylmethacrylate (PMMA) polymers, bis-GMA polymers, polyester polymers, polyacrylate polymers, polyvinyl polymers, epoxy polymers, and combinations thereof.
Statement 32. A method according to any one of Statements 28-31, where the polymeric gel is a methylcellulose gel.
Statement 33. A method according to any one of Statements 28-32, where the plurality of microspheres are polylactic glycolic acid microspheres.
Statement 34. A method according to any one of Statements 28-32, where the plurality of microsphere(s) further comprise one or more drugs, one or more peptides, one or more growth factors, one or more nucleic acids, or the like, or combinations thereof.
Statement 35. A method for treating an individual afflicted with or suspected of having drug-induced gingival overgrowth (DIGO) comprising: (a) obtaining a scan, mold, or model of the individual's oral cavity where a device will fit; (b) fabricating the device corresponding to the individual's scan, mold, or model of the oral cavity with a polymer; (c) generating the composition according to any one of Statements 1-13; (d) coating the device with the composition; (e) administering the completed device to the individual for sufficient time to treat the DIGO; and (f) optionally, monitoring the individual's response to the device treatment.
Statement 36. A method according to Statement 35, where the device is a dental prosthesis.
Statement 37. A method according to Statement 36, where the dental prosthesis is chosen from a splint, mouth guard, full denture, partial denture, and filling.
Statement 38. A method according to Statement 35-37, where the polymer is chosen from polymethylmethacrylate (PMMA) polymers, bis-GMA polymers, polyester polymers, polyacrylate polymers, polyvinyl polymers, epoxy polymers, the like, and combinations thereof.
Statement 39. A method according to any one of Statements 35-38, where the polymeric gel is a methylcellulose gel.
Statement 40. A method according to any one of Statements 35-39, where the plurality of microspheres are polylactic glycolic acid microspheres.
Statement 41. A method according to any one of Statements 35-40, where the plurality of microsphere(s) further comprise one or more drugs, one or more peptides, one or more growth factors, one or more nucleic acids, or the like, or combinations thereof.
Statement 42. A method for treating an individual afflicted with or suspected of having drug-induced gingival overgrowth (DIGO) comprising administering to the individual the composition according to any one of Statements 1-13 for a sufficient time to treat the DIGO.
Statement 43. A method according to Statement 42, where the polymeric gel is a methylcellulose gel.
Statement 44. A method according to Statement 42 or Statement 43, where the plurality of microspheres are polylactic glycolic acid microspheres.
Statement 45. A method according to any one of Statements 42-44, where the plurality of microsphere(s) further comprise one or more drugs, one or more peptides, one or more growth factors, one or more nucleic acids, or the like, or combinations thereof.

The following examples are presented to illustrate the present disclosure. They are not intended to limiting in any matter.

### EXAMPLE 1

This example provides a description of methods and materials of the present disclosure.

Cell Culture. Normal human oral keratinocytes (NOKSI) and fibroblast (HOF) cells were maintained in Dulbecco minimal Eagle's medium (Sigma-Aldrich, St. Louis, MO, USA) supplemented with 10% fetal bovine serum (Atlas Bio-logicals, Fort Collins, CO, USA) and 100 units/mL penicillin and 100 μg/mL streptomycin (LifeTechnologies, Carlsbad, CA, USA). Cells were grown in incubator at 37° C. in a humidified chamber with 5% CO₂.

Drugs treatment in cell culture. The cells were treated alone or with different combination of lipopolysaccharide, cyclosporine, phenytoin, and folic acid. All of them were purchased from Sigma-Aldrich (St. Louis, MO, USA). Each of them were used from 0 μg/ml to 40 μg/ml and the optimum concentration were found 10 μg/ml.

Western blot analysis. Cells were seeded in 60 mm plates with a density of 5×10⁵ cells per plate and washed briefly with Dulbecco's phosphate-buffered saline (Gibco, Waltham, MA, USA). Whole cell lysates were prepared using RIPA lysis buffer containing protease inhibitor cocktail (Sigma-Aldrich). Total protein concentration was determined using the BCA reagent (Pierce, Waltham, MA, USA). 20 μg of total protein were resolved on sodium dodecyl sulfate-polyacrylamide gel electrophoresis and transferred to polyvinylidenedifluoride membrane and immunoblotted. The blots were incubated for 1 h in 1% bovine serum albumin (BSA) (Sigma-Aldrich) to block the non-specific bindings followed by over-night incubation in primary antibodies (MyD88 and pP65 from Cell Signaling Technology, and human LITAF ThermoFisher Sigma) at 4° C. on shaker. Next day, blots were washed for three times (10 min each) in Tris-buffered saline containing 1% Tween20 (TBST) followed by incubation with secondary antibody (Goat anti-rabbit IgG HRP) with 1:10,000 dilution for 1 h. Following washes, bands were visualized using gel documentation Imaging Systems (BioRad). The band intensities were quantified using software ImageJ (NIH, USA) and relative protein levels were normalized by β-actin (Cell Signaling). To perform the western blot of animal tissue samples, the gingival tissues were collected from both BABLB/c and C57BL/6 mice studies. The soft tissues were separated from the mandibular bone and then crunched. The Bradford assay was performed to measure the protein concentration of each sample. After normalization, 20 μg of whole tissue protein lysate were proceeded for western blot analysis.

Enzyme linked immunosorbent assay (ELISA). ELISA was conducted with a mouse TNF-α ELISA Kit (PeproTech, USA) and TGF-β Emax ImmunoAssay System (Promega USA) to detect the TNF-α and TGF-β respectively. After normalization of the total cells or tissue protein lysates, 20 μg protein of each sample was used for the detection of TNF-α or TGF-β. ELISA was performed according to the manufacturers' instructions. For TNF-α, 100 ng/100 μl of capture antibody was added to each well of 96 well assay plate and incubated for overnight at room temperature. Blocking buffer of 200 μl per well were added to each well and incubated for 1 hour at room temperature. 20 μg of each protein sample as well as TNF-α standard (from 2 ng/ml to 0 ng/ml) were added and incubated the plates for 2 hours. 50 ng/100 μl of detection antibody was added in each well and incubated for 2 hours. Avidin-HRP conjugated antibody with a dilution of 1:2000 was added for 30 minutes. 100 μl per well of ABTS solution was added to each well and incubated until color change. Optical absorbing values were read at 405 and 450 nm and corrected by 650 nm. To detect the TGF-β, the ELISA plates were coated with TGF-β monoclonal antibody and incubated for overnight at 4° C. TGFβ 1×buffer were added to block the non-specific bindings at 37° C. for 35 minutes without shaking. The whole protein lysates or TGF-β standard (0-1,000 pg/ml) were added followed by Anti-TGFβ1 polyclonal antibody and incubated for 1 and 2 hours respectively. The samples were incubated with TGF-β HRP Conjugate for 2 hours at room temperature. TMB One Solution was added for 15 minutes and the reaction were stopped using 100 μl of 1N hydrochloric acid and the absorbance at 450 nm were recorded on a plate reader. Data were analyzed using excel software. At each step the sample were washed with PBST or wash buffer for four times using 200 μl per well.

Immunostaining and fluorescence microscopy. The cultured cells were fixed with 4% paraformaldehyde followed by permeabilization with phosphate buffered saline (PBS) containing 0.1% Triton X-100 (PBS-T), and blocking with PBS-T containing 5% bovine serum albumin (BSA). Then, the cells were incubated with anti-pP65 antibody (Cell Signaling, USA) followed by Alexa Fluor 488 conjugated-goat anti-rabbit secondary antibody (Invitrogen, Grand Island). Rhodamine phalloidin (Invitrogen) and 4′,6-diamidino-2-phenylindole (Vector labs, Burlingame) were added to visualize the actin and nucleus respectively. The samples were mounted and examined using a Zoe microscope (BioRad, USA). The image analyses were performed using the ImageJ (NIH) software.

Flow cytometry. NOKSI cells were seeded in 100 mm plates with a density of 1×10⁶ cells per plate. Cells were treated with drugs, LPS, and folic acid alone or in combination. After NOKSI were detached using Corning Cell Lifter (Corning) and 10 mM EDTA in PBS, the cells were washed with FACS buffer (PBS containing 1% BSA [Sigma] and 2 mM EDTA). Cells were stained by immunofluorescence for CD282 (TLR2) and CD284 (TLR4). Fluorescent labeled antibodies at a dilution of 1:1000 in staining buffer with 2% FBS in PBS were added and incubated for 30 min in dark on ice. Cells were centrifuged and washed thrice in staining buffer and passed through a 70 μm cell strainer before data acquisition. Data analysis was performed using FlowJo (TreeStar, Ashland, OR, USA). All incubations were performed on ice.

Reporter assay. The TNF-α promoter deletion constructs, Δ−1220 (−1220 to +65) and Δ−450 (−450 to +65) were synthesized from SwitchGear Genomics (USA) for the luciferase reporter assay. The human TNF-α promoter constructs or an empty pLightSwitch promoter reporter vector as a control was transfected in to NOKSI using the FuGene reagent (SwitchGear Genomics, USA). Cells were seeded with a density of 5×10³ per well of 96-well plate. GoClone reporter constructs and transfection reagents were mixed with optimum combination and incubated for 30 minutes. The media were changed to the seeded cells and reaction mixture were gently dripped onto the seeded cells, shaked the plate gently and incubated for next 24 hours. The LightSwitch assay reagents were added to the transfected plates and incubated for 30 minutes at room temperature. Luciferase activity were measured with with a luminometer (Wallac Vector3; PerkinElmer, Boston, MA).

Chromatin Immunoprecipitation (ChIP) Assay. ChIP assays were performed using a SimpleChIP Plus Enzymatic Chromatin IP kit (no. 9005; Cell Signaling Technologies). NOKSI were cultured with density of 1×10⁶ cells per 100 mm plate. Cells were treated with drugs, LPS and folic acid alone or in combinations for next 24 hours. The cells were cross-linked with 1% formaldehyde and quenched the reaction using 0.125 M glycine at room temperature for 10 min. Under cold condition, the fragmented chromatins were treated with nuclease and subjected to sonication. Chromatin immunoprecipitation was performed with rabbit anti-LITAF antibody (ThermoFisher, USA), rabbit anti-histone H3 (a positive control; 1:50) (Cell signaling, USA), and normal rabbit IgG (a negative control) (Cell signaling, USA). After reverse cross-linking DNA fragments were extracted and purified with a DNA purification kit (Quagen). Immunoprecipitated DNA was quantified by quantitative real-time PCR using SYBR green (Applied Biosystems). The sequences of the primers for LITAF binding sites in the TNF-α promoter are shown in the figures. Fold enrichment was calculated based on the threshold cycle (CT) value of the IgG control using the comparative CT method.

Microsphere Fabrication. The folic acid was encapsulated in poly (lactide-co-glycolide) (PLG) (8515DLG7E, Lakeshore Biomaterials, Birmingham, AL, USA) microspheres using double emulsion solvent evaporation method. Briefly, a 100 μL quantity of proteins dissolved in molecular-grade water was pipetted into 1 mL of 5% PLG in ethyl acetate (Sigma-Aldrich Corp., St. Louis, MO, USA) and sonicated (Sonics and Materials Inc., Newtown, CT, USA) on 50% magnitude for 1 min. A second emulsion composed of 1% polyvinyl acetate (PVA) (Sigma-Aldrich) and 7% ethyl acetate was then added and vortexed for 15 sec. The solution was then added to a 1% PVA solution with continuous stirring for 3 hrs at room temperature and filtered through a 0.2-μm filter (Nalgene, ThermoFisher Scientific, Rockville, MD, USA), collected by centrifugation at 2,000 rpm for 10 min (Eppendorf, Hamburg, Germany), freeze-dried for 16 hrs (Labconco, Kansas City, MO, USA), and stored at −20° C. The microspheres fabricated with this technique were between 2 and 200 microns.

Scanning electron microscopy. For scanning electron microscopy (SEM) analysis, the microspheres were attached to the adhesive tape surface for strong holding. The samples were sputter coated with gold using Ion sputter “Au—Pb” target for 120 seconds and examined using scanning electron microscopy (Hitachi, S-4700, Japan), at an accelerating voltage of 15 kV.

Gel preparation and folic acid release study. Folic acid (100 μg/ml) (Sigma-Aldrich, St Louis, MO) was encapsulated in PLG microspheres as described above. The resulting lyophilized powder was formulated into an oral gel containing 20 mg of folic acid microspheres mixed with 5% of methylcellulose (Sigma-Aldrich, St Louis, MO) in 330 μl of phosphate buffer saline. The gels were prepared fresh daily before applications and repeated until end of the study period (14 days).

Animal Study. Ligature-induced inflammation models have been previously described. However, conventional in-vivo gingival overgrowth models in mice to utilize a molar ligature is technically arduous and difficult to standardize. Therefore, we developed a novel in-vivo anterior ligature model to develop DIGO. We examined two strains of mice, BALB/c and C57BL/6 were used. Sterile ligature (5-0 Silk thread) were placed around the lower central incisors on both sides in mice for 14 days at anaesthetized conditions using ketamine and xylazine. Mice were daily checked for placement of suture. Cyclosporine 50 mg/kg/body weight or vehicle were injected intraperitoneal daily until day 14. The vehicle used to dissolve the drug was 10% ethanol and 10% polyethyleneglycol castor oil (Sigma-Aldrich, St. Louis MO) in sterilized distilled water to a volume of 100%. All mice were scarified at day 14. Euthanasia was performed using carbon dioxide followed by cervical dislocation.

The study of the BALB/c mice consisted of five groups namely; negative control (no disease), disease group (Ligatures and Cyclosporine), Preventive treatment group: (ligature, cyclosporine and Folic acid (PO and Local).

In the combined prevention and treatment group, both modes of folic acid treatments were provided including PO and local gel application. The folic acid gel preparation applied onto the ligature surface around the mandibular incisor from day 0 together at the start of the cyclosporine IP injections.

Using the C57BL/6, the DIGO were induced in 33 male mice for 14 days in an anaesthetized condition as described above. The study of C57BL/6 consisted of 5 groups namely;
1—Negative Control
2—Disease: Ligature+Cyclosporine (PO).
3—Prevention: Ligature+Cyclosporine with Folic acid (PO).
4—Active Treatment: Ligature+Cyclosporine with Folic acid (Local).
5—Prevention and Treatment: Ligature+Cyclosporine with Folic acid (PO and Local).

In the prevention group, folic acid (10 μg/ml) was included in the drinking water from the time of ligature placement until end of the study (14 days). In the active treatment group, folic acid gel preparation applied onto the ligature surface around the mandibular incisor from day 0 at the initiation of the cyclosporine injections. In the combined prevention and treatment group, both modes of folic acid treatments had provided including PO and local gel application as described above.

MyD88 Knockout mice. Further, wild type and MyD88 knock-out mice were used to confirm our DIGO study. In this study, both male and female were included from wild type as well as from knockout mice. The sterile ligature (5-0 Silk thread) were placed around the lower central incisors of the Wild type (3 Males and 7 Females) and Myd88-Knock-out (14 Males and 11 Females) mice for 14 days. This study consisted of 3 groups namely;
1—Negative Control
2—Disease (Wild type): Ligature+Cyclosporine (PO).
3—Disease (Myd88 Knocked-out): Ligature+Cyclosporine (PO).

Macroscopic Analysis. Mandibles were dissected and images of the gingival occlusal, buccal and lingual surfaces were obtained using stereomicroscope. Then the mandibles divided into two halves and subjected to the western blot and Immunohistochemistry evaluation.

3D digital imaging. The mandibles were digitized with an intraoral 3-dimensional scanner (TRIOS; 3 Shape, Copenhagen, Denmark). The digitized casts were sent to the computer-aided design and computer-aided manufacturing (CAD-CAM) software. The images were transferred to standard tessellation language (STL) files and analyzed quantitatively. The surface area and volume of the tissues were extracted from 3D images using ImageJ software.

Histopathology and Immunohistochemistry. Mice mandibles were fixed in 10% formaldehyde for overnight at 4° C. on shaker with 45 rpm and then the solution were replaced with 70% ethanol. The tissues were decalcified with 10% ethylenediaminetetraacetic acid (EDTA) for 1 week and were dehydrated through graded ethanol, cleared with xylene, and embedded in paraffin. 4 μm serial sections were cut in a buccal-palatal direction and stained with Hematoxylin and Eosin (HE) and observed under light microscopy for histomorphological evaluations and quantitatively analysis was performed using ImageJ software.

For immunohistochemistry, unstained sections were deparaffinized in xylene for 10 minutes and then transferred to ethanol for 20 minutes. Antigen retrieval was performed by boiling the deparaffinized sections in 10 mM citrate buffer (pH 6.0) using a microwave oven for 3 minutes at highest power 10. The sections were allowed to cool at room temperature and washed twice in TBST for 5 minutes each. Sections were further treated with 2% hydrogen peroxide in PBS for 10 minutes to block the endogenous peroxidase activity, followed by a TBST wash. Secondary antibody species serum (Vectastain Universal ABC-AP KIT (PK-6200)) in PBS was used to block the background staining for 1 hr. The sections were then incubated for overnight at 4° C. with primary antibodies against selected molecular markers such as Myd88, LITAF, and pP65. The optimal concentration of individual antibodies Myd88, LITAF, and pP65 varied greatly and were 20 μg/ml, 5 μg/ml and 1 μg/ml respectively. This was followed by Biotinylated (1 μg/ml) secondary antibody for 30 min and TBST washing 2 times for 5 minutes each. The Avidin-Biotin solution was added and incubated for 30 min and repeated the washing step. The samples were incubated in ImmPACT DAB EqV (Chromogen and peroxidase) as a chromogenic substrate, counterstained and mounted for histological examination.

Human tissues samples. The samples were de-identified, and clinical tissue sections were procured from academic institute archives at Department of Oral and Maxillofacial Pathology, KLE VK's Institute of Dental Sciences, Belgaum, India. However, these cases have a known medical history for prescribed drug intake that are known to induce gingival overgrowth. These patients presented clinically with DIGO and were biopsied for histopathological examination. Histological sections of patient's tissues who have gingival overgrowth induced by medication were de-identified and archived. Sections were stained with routine histological stains including hematoxylin and eosin, Mason trichrome and Picrosirius Red to study the tissue architecture. The immunostaining method were used same as above.

Statistical analysis. Data are presented as the mean and standard deviation from the repeated experiments. Statistical analysis was performed using Student's t-test. The difference between groups were considered significant when the p value was <0.05.

### EXAMPLE 2

This example provides description of methods of making and using a composition of the present disclosure.

Animal study. Prior mouse models of DIGO involve placement of ligatures around the molar that is technically arduous and difficult to standardize. Therefore, this project sought to develop a novel anterior ligature model by placing sutures in the anterior mandibular region. It should be emphasized that this model is more appropriate to the human clinical disease presentation that predominantly affects the anterior gingiva. Two strains of mice BALB/c and C57BL/6j males at 4 weeks of age were procured from Charles River Laboratories, Wilmington MA. Transgenic animals for MyD88 knockout mice on a C57BL/10Xm strain were originally procured from Jackson Laboratory, Bar Harbor, ME and the colony were expanded by Dr. Jill Kramer, Oral Biology, University at Buffalo who provided these animals for this study. All animal procedures were approved by the Animal Care and Use Committee, University at Buffalo.

Study Design. Three animal studies were performed and are individually described below.

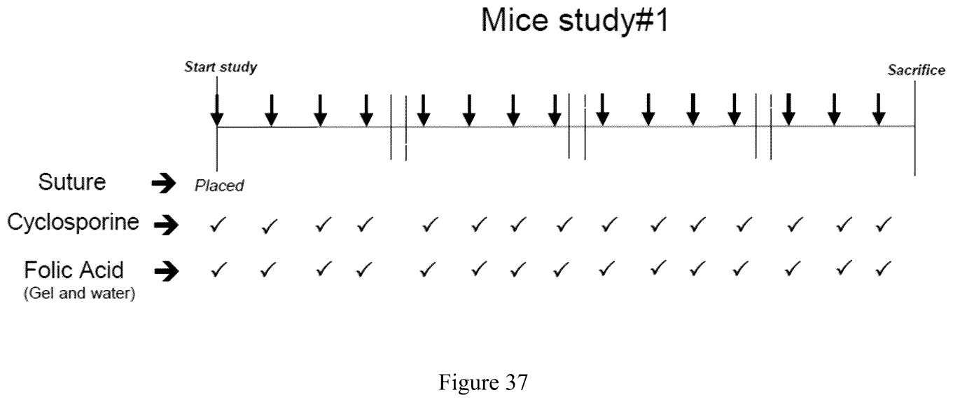

Mice study #1: Pilot study to establish DIGO disease model. Sterile sutures (5-0 Silk thread, Ethicon, Johnson & Johnson, Somerville, NJ) were placed around the lower central incisors on both sides in BALB/c (Male) mice anaesthetized conditions using Ketamine (90-100 mg/kg bodyweight) and Xylazine (5-10 mg/kg body weight) using a 0.1 cc syringe with a 25-gauge needle intraperitoneally (FIG. 37). Mice were checked daily for intact suture placement and replaced when necessary. Cyclosporine (50 mg/kg/body weight) or vehicle was injected intraperitoneal daily until day 28. The controls were injected with the vehicle used to dissolve the drug, which was 10% ethanol and 10% polyethylene glycol castor oil (both Sigma-Aldrich, St. Louis MO) in distilled water. For preventive treatments, Folic acid was supplemented in the drinking water (10 μg/ml) ad libitum and methylcellulose gel with the Folic acid (100 μg/ml) microspheres was applied daily on the anterior gingiva. The three groups in this study were:
1. Control group (n=1): no interventions were performed
2. Disease group (n=4): placement of sutures and cyclosporine injections (i.p.).
3. Prevention group (n=4): placement of suture, cyclosporine and folic acid (p.o. & local).
   Treatments were continued for 28 days and digital images were captured over the time course.

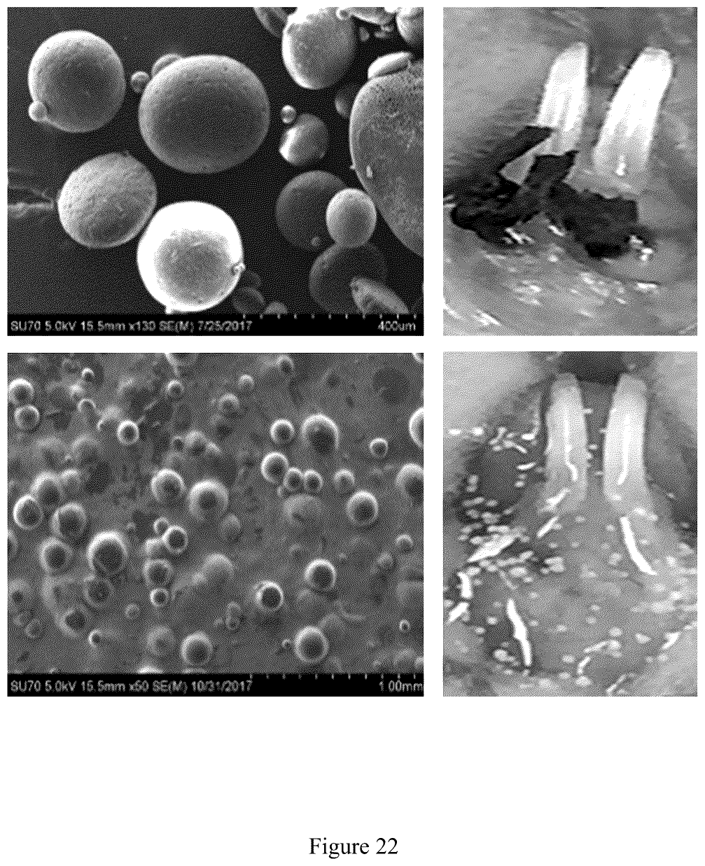

Mice study #2: Validation study for preventive and active treatment for DIGO. Following the successful establishment of the DIGO mouse model that showed a prominent phenotype at 14 days, the second mice study was performed to examine both prevention and active treatment with folic acid. For the active treatment group, DIGO (suture placement and cyclosporine injections) was allowed to progress for 1 week and then sutures were removed and cyclosporine injections were stopped for the remainder of the study in two groups of mice (FIG. 23). One set of these mice were used as a control disease group (FIG. 23A) while the other group was treated with folic acid (gel and in drinking water) (FIG. 23B). In the prevention and treatment group, folic acid (10 μg/ml) (Sigma-Aldrich, St. Louis MO) was included in the drinking water from the time of suture placement until end of the study (14 days). Additionally, gel applications were repeated daily until the end of the study (14 days). The four groups in this study were:
1. Control group (n=2): no interventions were performed
2. Disease group (n=12): placement of sutures and cyclosporine injections (i.p).
3. Prevention group (n=13): placement of suture, cyclosporine and folic acid (p.o. & local).
4. Treatment group control (Disease) (n=3): placement of suture and cyclosporine 1 week only
5. Treatment group (n=3): placement of sutures, cyclosporine 1week, then folic acid (p.o. & local).

Mice study #3: Transgenic mice study to validate the role of MyD88 in DIGO. In the final study, we used wild-type and MyD88 knock-out mice to confirm a role for MyD88 mediating DIGO as described above. Briefly, sterile sutures (5-0 Silk thread, Ethicon, Johnson & Johnson, Somerville, NJ) were placed around the lower central incisors on both sides in C57BL/10Xm (Male and Female) mice anaesthetized conditions using Ketamine (90-100 mg/kg body weight) and Xylazine (5-10 mg/kg body weight) using a 0.1 cc syringe with a 25-gauge needle intraperitoneally. Mice were checked daily for intact suture placement and replaced when necessary. Cyclosporine (50 mg/kg/body weight) or vehicle was injected intraperitoneal daily until day 14. The three mice groups in this study were;
1. Control (n=3)
2. Disease (Wild-type, n=10, 3 Males and 6 Females): suture and cyclosporine (i.p)
3. Disease (Myd88^{-/-}, n=15, 5 Males and 10 Females): suture and cyclosporine (i.p)

Sacrificing and mandible dissection. Animals were sacrificed at the end of the study by Carbon dioxide asphyxiation and cervical dislocation. Mandibles were dissected and processed for imaging or molecular analyses.

Digital photos. Dissected mandibles were photographed using a digital camera (iPhone 6S Plus, 12 megapixels with optical image stabilization).

2D planimetry with stereomicroscopy. For further evaluations of the samples, digital images were captured of the dissected mandibles using stereomicroscope (Nikon SMZ1000) to assess the gingival overgrowth by 2D planimetry.

3D Digital Imaging and Volumetric Analysis. A 3Shape TRIOS® intraoral digital scanner was used to scan all of the dissected tissue and create a 3D STL (STereoLithography) file from each scan. Autodesk Meshmixer® (Autodesk 23 Inc., CA, USA) was used to overlay each individual scan on top of one another, keeping each of the distinct mice studies separately. Anatomical landmarks of the mandibular incisors, interdental papilla, and molars were used to make sure scans were accurately overlaid. Autodesk Meshmixer® was then used to selectively remove the mandibular incisors from each digital scan. The scans were all saved and imported into Autodesk Netfabb® (Autodesk Inc., CA, USA) where plane cuts were used to simultaneously crop all scans to include only the essential parts of the gingival tissue that would indicate an increase or decrease in growth. The cropped scans were saved and imported into open source software platform 3DSlicer 4.8, where they were volumetrically analyzed using the “Volumes” module. The quantified volume values were statistically analyzed in GraphPad Prism® 7 (GraphPad Software Inc., CA, USA).

Human Research Protocol. An institutional review board (IRB) proposal was submitted through the online portal, CLICK, to the University at Buffalo to seek human clinical samples with DIGO. De-identified samples were procured from academic institutional archives from dental schools in India including Oral and Maxillofacial Pathology department at KLE VK's Institute of Dental Sciences and Maratha Mandal Institute of Dental Sciences, Belgaum, India. All cases had been diagnosed as DIGO with a known medical history for prescribed drug intake. These patients had presented clinically with DIGO and had been biopsied for histopathological examination for confirmatory diagnoses.

Histopathology and Immunohistochemistry. Mice mandibles were fixed in 10% formaldehyde for overnight at 4° C. on the shaker with 45 rpm and then the solutions were replaced with 70% ethanol. The tissues were decalcified with 10% ethylenediaminetetraacetic acid (EDTA) for 1 week and were dehydrated through graded ethanol, cleared with xylene, and embedded in paraffin. 4 μm serial sections were cut in a buccal-lingual direction and stained with Hematoxylin and Eosin (HE) and observed under light microscopy for histomorphological evaluations.

For immunohistochemistry, unstained sections were deparaffinized in xylene for 10 minutes and then transferred to ethanol for 20 minutes. Antigen retrieval was performed by boiling the deparaffinized sections in 10 mM citrate buffer (pH 6.0) using a microwave oven for 3 minutes at highest power 10. The sections were allowed to cool at room temperature and were washed twice in TBST for 5 minutes each. Sections were further treated with 2% hydrogen peroxide in PBS for 10 minutes to block the endogenous peroxidase activity, followed by a TBST wash. Secondary antibody species serum (Vectastain Universal ABC-AP KIT (PK-6200)) in PBS was used to block the background staining for 1 hr. The sections were then incubated for overnight at 4° C. with primary antibodies against selected molecular markers such as TLR2/4, Myd88, LITAF, NFκB, TNF-α, and TGF-β. The optimal concentration of individual antibodies Myd88, LITAF, NFκB varied greatly, 20 μg/ml, 5 μg/ml, and 1 μg/ml respectively. This was followed by Biotinylated (1 μg/ml) secondary antibody for 30 min then TBST washing 2 times for 5 minutes and Avidin-Biotin solution for 30 min then the samples were washed with TBST for 2 times 5 minutes each. The samples were incubated in ImmPACT DAB EqV (Chromogen and peroxidase) as a chromogenic substrate, counterstained with hematoxylin (Sigma-Aldrich MHS 16-500 ML) and mounted with DPX Mountant for histology (BCBR1545V) for histological examination.

Western blot. Gingival tissues were collected from all groups of the 1st and 2nd mice studies. The soft tissues had separated from the mandibular bone and then crunched. The Brad-ford assay was performed to measure the protein concentration of each sample. After normalization, 20 μg of whole tissue protein lysate was mixed with LDS western blot sample buffer (4×) and boiled for 10 minutes at 100° C. The western blot analyses were performed to detect Myd-88, LITAF, and NFκB. The samples were run on SDS polyacrylamide gel and transferred to PVDF membrane. After 60 min incubation in blocking buffer (1% BSA in TBST) with gentle shaking, the membranes were incubated for overnight with the following optimal dilutions of primary antibodies in blocking buffer: 1 in 50 dilutions for rabbit anti-mice Myd88, 1 in 200 for rabbit anti-mice LITAF, and 1 in 100 for rabbit anti-mice NFκB. After washing in TBST (1% Tween 20 in PBS) for 3 times, 5 minutes each, the membranes were incubated for 1 h in 1 into 5000 dilutions of the HRP conjugated secondary antibody. The samples were 3 times with TBST for 5 minutes each. Anti-β-actin HRP-conjugated mouse monoclonal IgG1 antibody (Cell signaling, USA) was used for the loading control. The band intensities were measured by the software ImageJ (NIH, USA), and relative protein levels were normalized by β-actin.

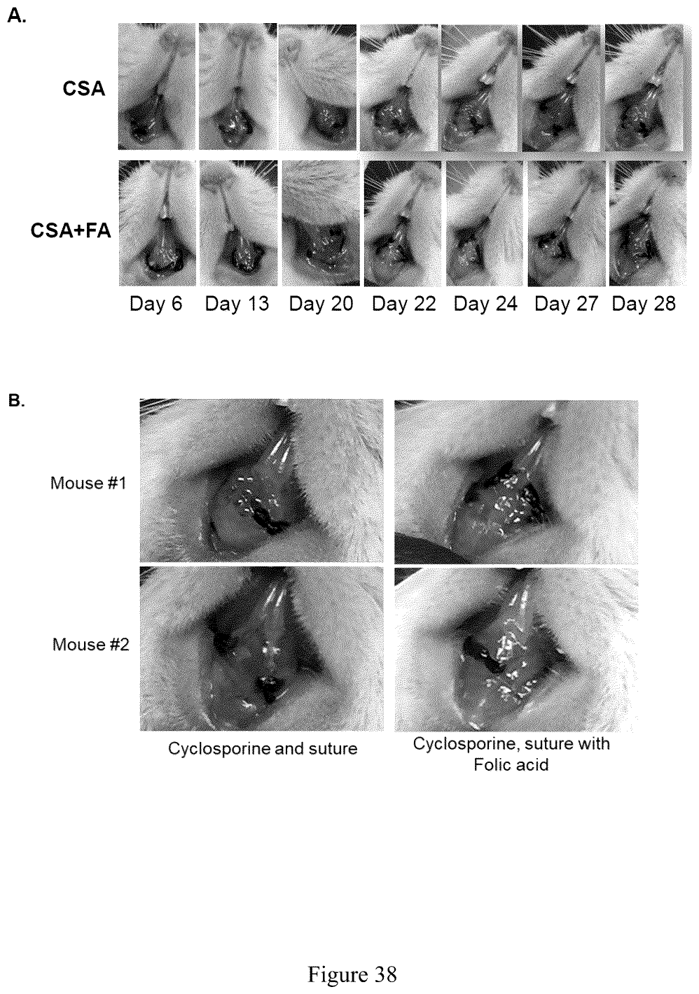

Mice study #1: Pilot study to establish DIGO disease model. The pilot study was performed in BALB/c mice that noted increased gingival overgrowth in the cyclosporine (disease) group on the 6^{th} day and was more prominent over the course of the study up to 28 days (FIG. 38A). These overgrowths demonstrated erythema, inflammation, and ulceration in different areas. In contrast, the folic acid group demonstrated less swelling and inflammation that became apparent at day 8 (FIG. 38B). The reduction in DIGO in the FA group was clear throughout the course of the study as evidenced by the clinical images (FIG. 38B). A major outcome of this study was that DIGO in mice was clearly evident by 14 days and therefore future studies could be performed in a shortened time course.

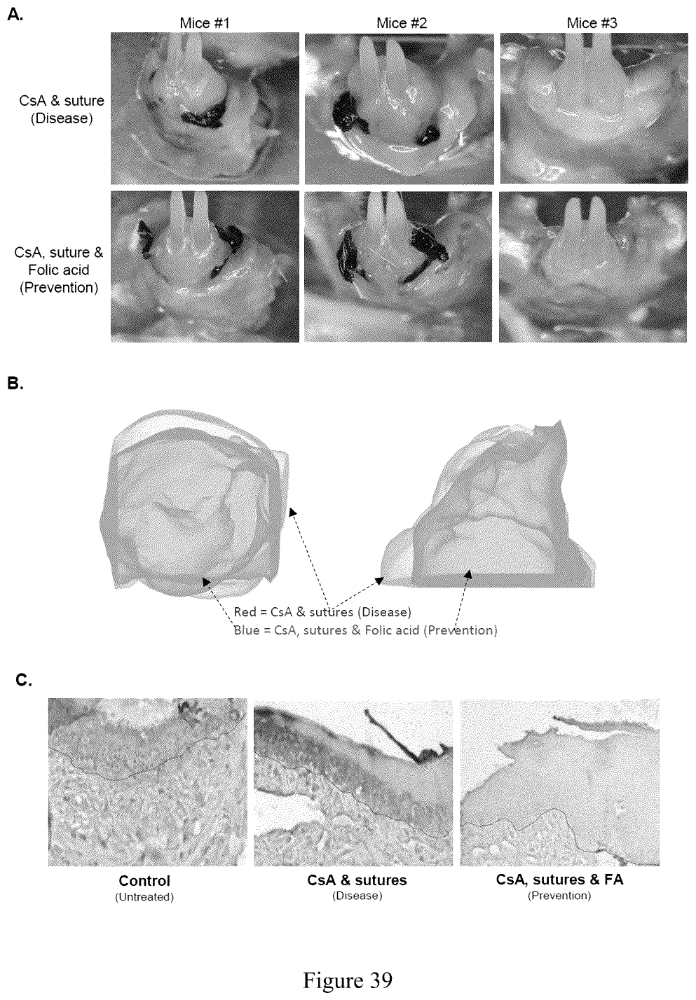

2D planimetry with stereomicroscopy. For further evaluations of the samples, we took images of the dissected mandibles using stereomicroscope (Nikon SMZ1000) to assess the gingival overgrowth in the samples of the disease and treated groups. After the assessment, we observed that the three mice samples of the disease group showed more gingival overgrowth compared to the prevention group. Furthermore, the 2^{nd} mice in the disease group showed the most prominent DIGO in comparison to the 1^{st} and 3^{rd} mice (FIG. 39A).

3D volumetric analysis of gingival overgrowths. Since calculating the gingival overgrowth in the stereomicroscopic images using IMAGEJ were not possible, due to the anatomical features and curvatures of the mandibles. Therefore, we scanned the dissected mandibles using the 3Shape TRIOS® intraoral digital scanner. After scanning of the mandibles, we created a 3D STL (Stereo Lithography) file from each scan and we overlaid each individual scan on top of one another, keeping each of the distinct mice studies separately. As it can be inferred from the overlaid of the disease group sample and prevention group sample revealed that the volume of the disease sample was significantly higher than the prevention sample (FIG. 39B). This finding suggested that folic acid treatments reduced gingival overgrowth compared to disease group.

Immunohistochemical analysis. In order to confirm the role of MyD88 in DIGO, we examined the histopathological stained sections with the MyD88 and LITAF antibodies to detect the antigens and to reveal their distributions in the pathological tissues. Digital images were captured using a Nikon Eclipse TE2000-U microscope equipped with Nikon Ds-Fi1 color camera (2560×1920 pixels) and Spot Advanced Software (4.0.4. version). Then, we examined the histopathological images of the mice model type BALB/c and we assessed the expressions of the Myd88 in the disease group in comparison to the negative control and Folic acid treated group. We observed that the Myd88 expression in the epithelium of disease group was significantly higher compared to the prevention and control groups. (FIG. 39C).

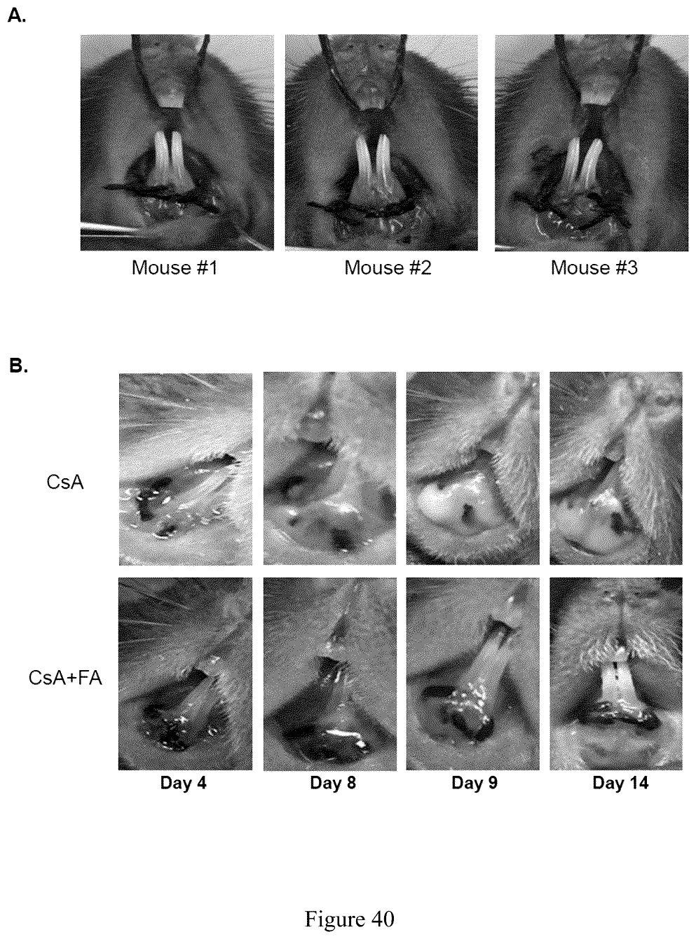

Mice study #2: Validation study for preventive and active treatment for DIGO. The second mice study in C57B/J6 mice was designed to validate the efficacy of the folic acid treatments as well as examine a treatment strategy. In the disease group, DIGO was evident by the 8^{th} day and prominent erythema and ulcerations of the enlarged gingiva were noted (FIG. 40A). The difference in the mice strains (BALB/C versus C57B/6J) did not appear to make a major difference. Preventive treatment with folic acid prominently reduced gingival overgrowth as evident in the clinical images (FIG. 40B). In the active treatment group, the gingival overgrowth appears to reduce following suture removal and stopping cyclosporine injections but overgrowth persisted until the end of the study (day 14). Folic acid treatments were noted to be efficacious in this group as well to effectively reduce gingival overgrowths.

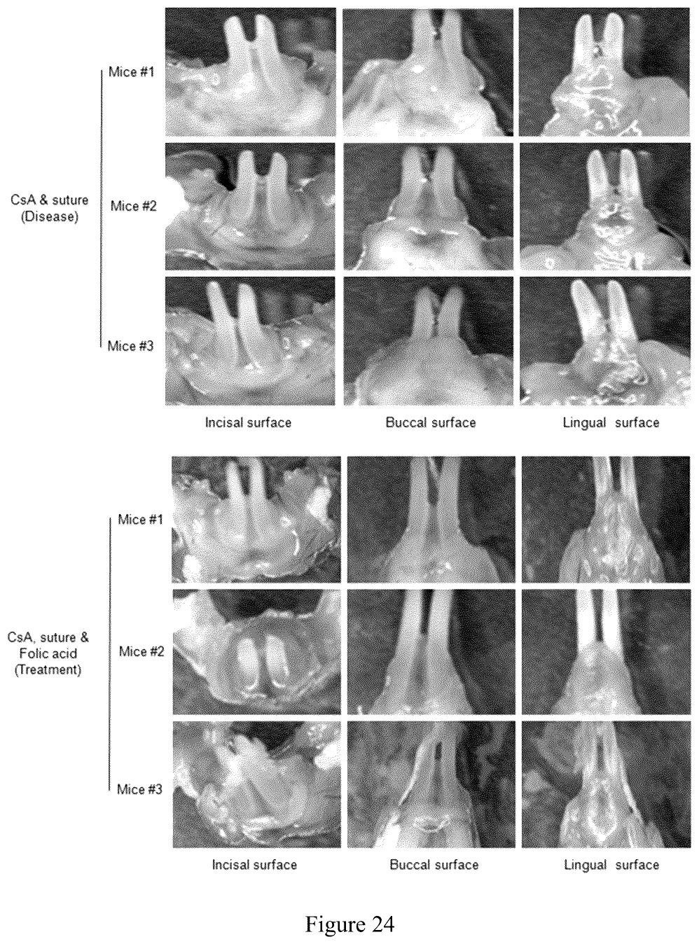

2D planimetry with stereomicroscopy. Analyses of the stereomicroscope images of the gingiva in these samples noted that the disease group demonstrated prominent gingival overgrowth (FIG. 25B) while the prevention group showed the minimal increase in gingival overgrowths (FIG. 25C) as compared to non-treated, control mice (FIG. 25A). It is prudent to point out that the gingival overgrowth appeared to be most prominent on the lingual compared to the buccal surfaces. A range of gingival enlargements was noted among the mice as noted in these images. Nonetheless, all mice appear to respond to the folic acid preventive treatments.

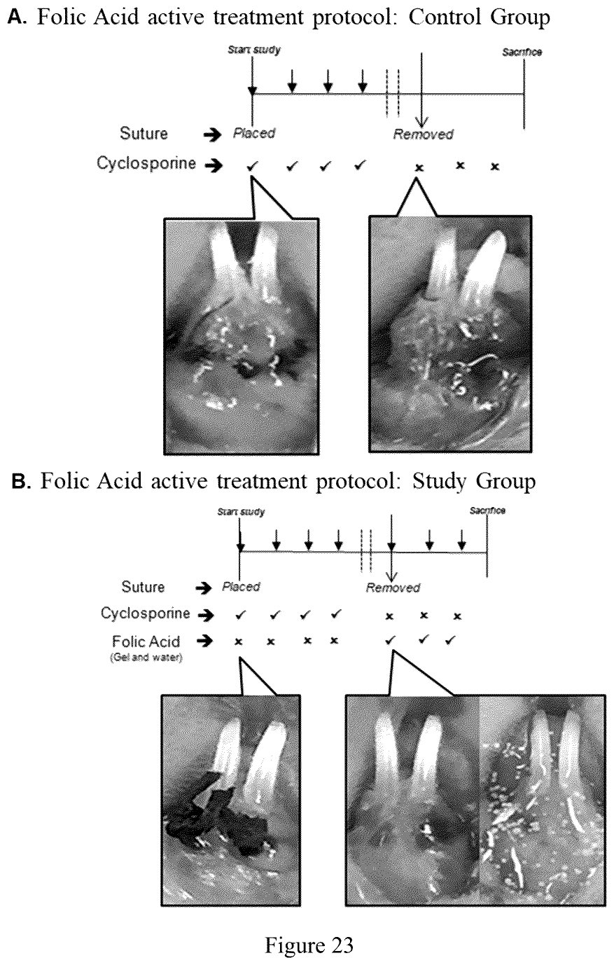

In the active treatment group that generated gingival overgrowth over 1 week with the placement of sutures and CsA treatments and removal, the gingival overgrowth appears to reduce somewhat over time (FIG. 24). The folic acid active treatment group appear to even further reduce these gingival overgrowths indicating the effectiveness of the treatment even after the establishment of the disease. On careful evaluation, we observed that they were no significant differences on the incisal, buccal or lingual surfaces of the dissected mandible between the two groups.

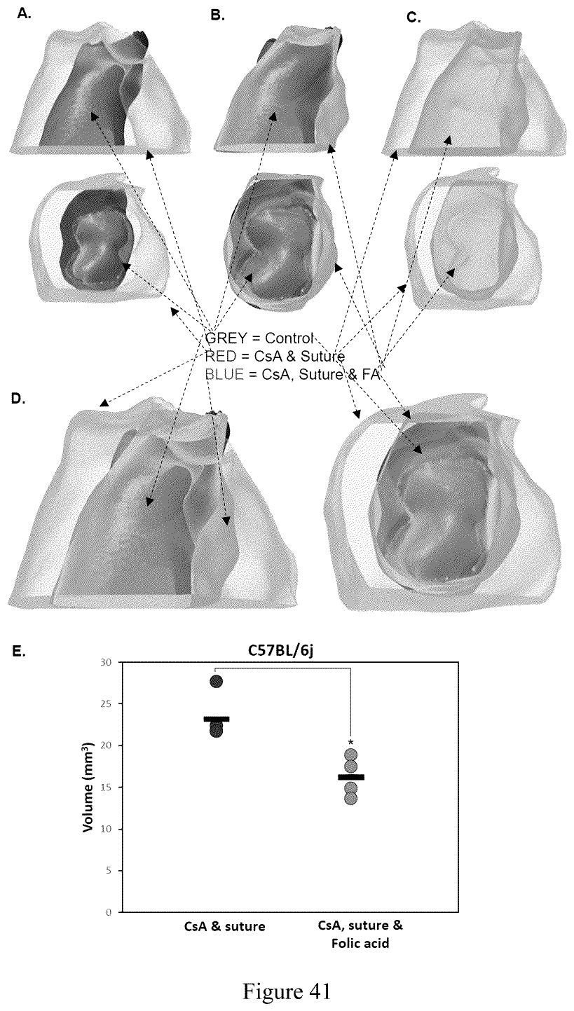

3D volumetric analysis of gingival overgrowths. As we did in the first experiment, we scanned the dissected mandibles using 3Shape TRIOS intraoral digital scanner and analyzed the gingival volumes. We observed that the overlaid 3D volumes of a representative control (untreated, grey) sample and disease sample showed a significant increase in the latter (FIG. 41A). On the other hand, the overlaid volumes of the control and folic acid treated showed little difference, with some increase apparent on lingual surfaces (FIG. 41B). Furthermore, the overlaid volumes of the disease and treated gingiva showed a significant difference (FIG. 41C). A composite overlay of all three representative highlights the significant differences in their apparent volumes (FIG. 41D). Quantitative analyses of the 3D morphometric analysis between the disease and treatment group revealed the approximate mean volumes to be 23.17±1.134 mm³ and 16.19±0.9224 mm³ respectively which were statistically significant (p<0.005) (FIG. 41E). These results validated the ability of folic acid as an effective treatment for DIGO.

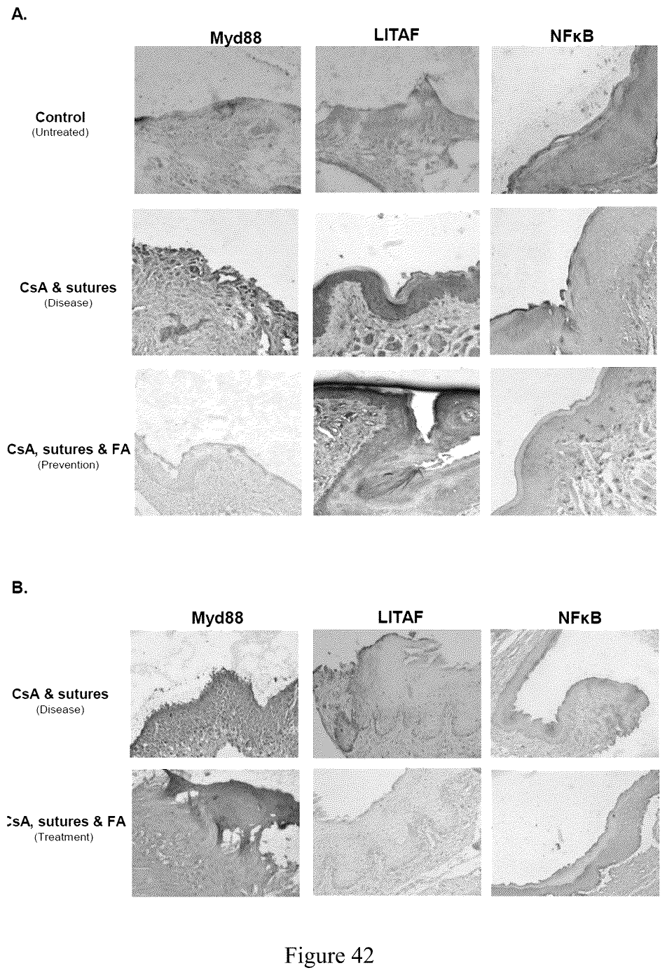

Immunohistochemical analysis. Digital images of representative histopathological sections were captured from control (untreated), disease and folic acid-treated mice gingival tissues subjected to immunostaining for Myd88, LITAF, and NFκB expression. The epithelial Myd88 expression in the disease group was significantly higher compared to both treated and control groups (FIG. 42A). Moreover, the LITAF expression in the epithelium was higher in the disease group and uniformly distributed compared to the control and treated groups. Generally, the histopathological examination of the three groups sample demonstrated that the Myd88 and LITAF epithelial expressions were higher in the disease group compared to the treatment group. However, NFκB epithelial expression appeared to be higher in the control group followed by the disease group. These patterns were consistent with the active folic acid treatment groups as well (FIG. 42B). Overall, these results were consistent with the ability of folic acid to modulate Myd88 expression in enabling its therapeutic responses in DIGO.

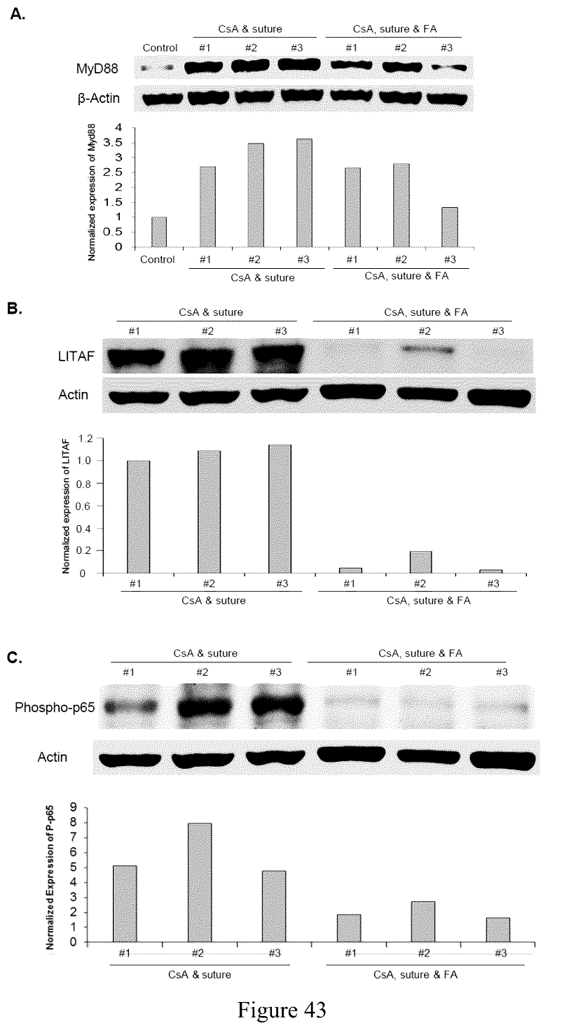

Immunoblotting of mice gingival samples. For further validation, we performed immunoblotting for Myd88, LITAF and phospho-p65 expression in three mice gingival tissue samples from all three groups. Our findings revealed that the disease group had higher Myd88 expressions compared to the control and treatment group (FIG. 43A). Both LITAF and phosphor p65 that are downstream of the Myd88 pathway demonstrated a similar pattern (FIGS. 43B and C). This analysis confirmed the role of folic acid in Myd88 mediated reduction of gingival overgrowth.

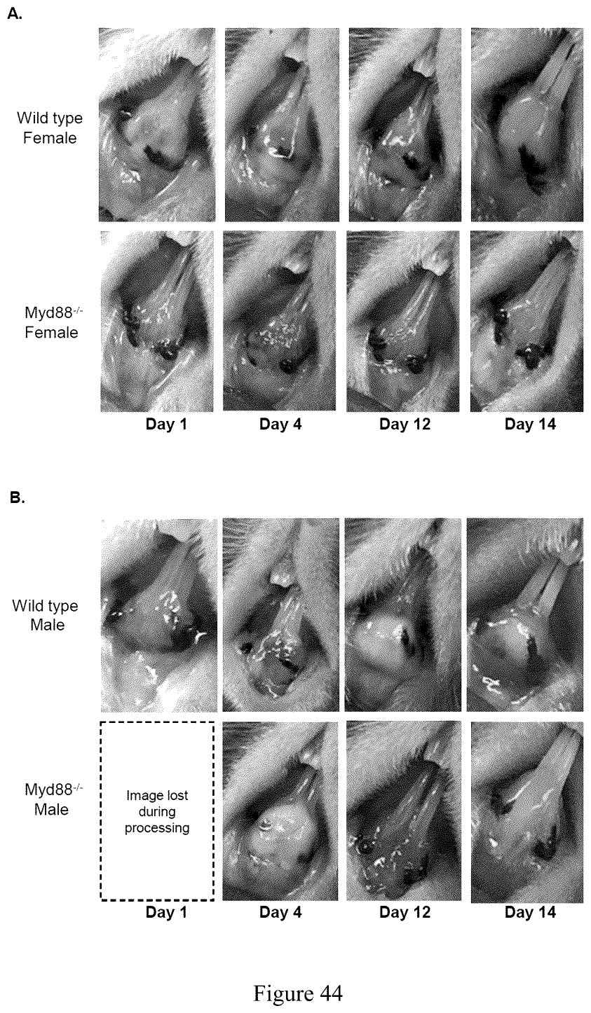

Mice study #3: Transgenic mice study to validate the role of MyD88 in DIGO. To confirm a potential role of MyD88 in mediating DIGO, we next examined a transgenic mouse model with homozygous deletion of exon 3 in a C57BL/10Xm strain. Approximately age-matched (4-6 weeks old) males and females were used in the study. Clinical images were taken over the 14 days. We observed there were reduced gingival overgrowths in the MyD88^{-/-} mice compared to their wild-type littermates (FIG. 44). The gingival overgrowth in the wild-type female became more severe over time while the MyD88^{-/-} female showed reduced swelling and inflammation (FIG. 44A). The wild-type male developed gingival overgrowth started on day 4 and increased over time until the end of the study on day 14 (FIG. 44B). In contrast, Myd88^{-/-} male demonstrated reduced gingival overgrowth at day 4 that was less prominent over the time course of the study.

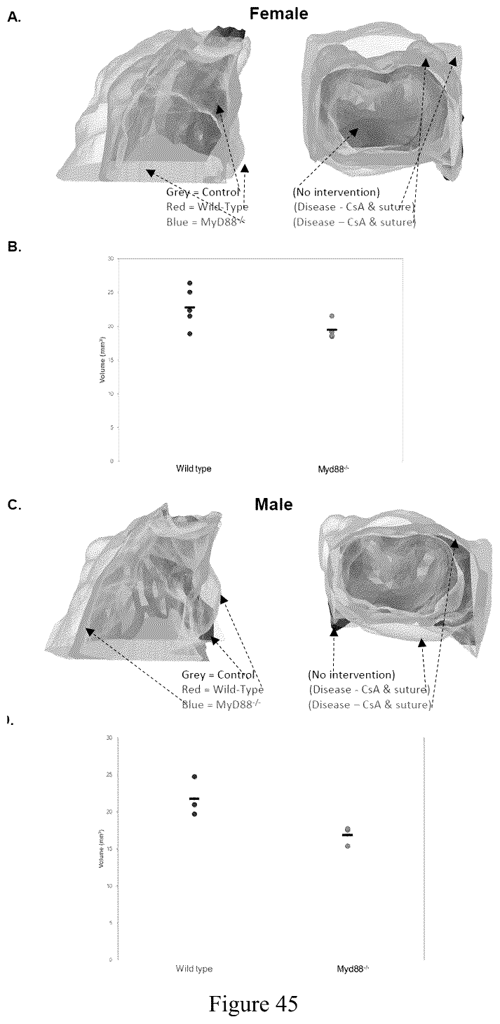

3D volumetric analysis of gingival overgrowths. Dissected mandibles of wild-type and Myd88^{-/-} mice with DIGO were examined using 3D digital analyses. The volumetric analysis revealed wild-type disease samples had higher gingival volume than the Myd88^{-/-}mice gingival samples in both genders (FIGS. 45A and C). Wild-type DIGO samples in females and males demonstrated the highest mean volumes among all groups with 23 mm³ and 22 mm³ respectively (FIGS. 45B and D). On the other hand, the mean volume of the Myd88^{-/-} females and males were 19 mm³ and 17 mm³, respectively (FIGS. 45B and D). These findings were noted to be statistically significant n=9/8 p<0.05.

Study with human patient samples. Human protocol approval. The UB IRB review deemed this protocol as non-human research as archived, de-identified samples were being requested. Following approval, nine human samples were shipped from India and received in the lab.

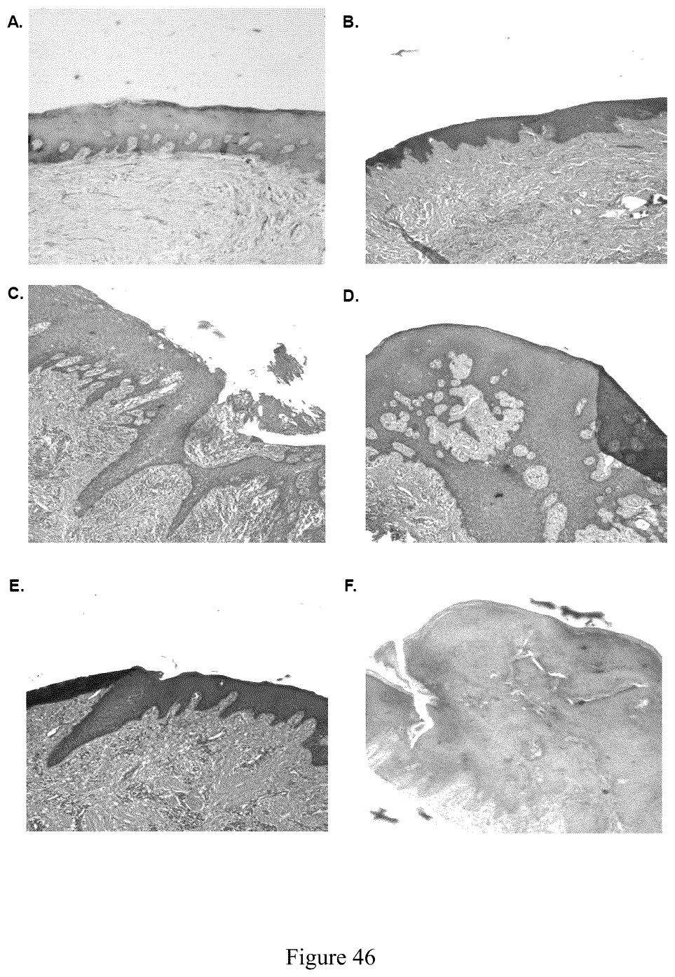

Histopathological analyses. The overall histological analysis of H & E stained DIGO human samples revealed mild hyperkeratosis, pseudoepitheliomatous hyperplasia with elongated rete-ridges, acanthosis, prominent fibrous stroma and few cells in the matrix (FIG. 46). Few inflammatory cells were evident in some of the samples that appear to be dependent on the stage of disease and additional, inadvertent trauma or irritation. In the section from patient #1 and 2, typical fibrotic features of DIGO are apparent including hyperparakeratosis, pseudoepitheliamatous hyperplasia, acanthosis and prominent fibrous stroma (FIGS. 46A and B). In sections from patient #3 to 5, features of epithelial hyperplasia and inflammatory infiltrate are prominent (FIG. 46C-E). Finally, in patient #6, prominent vascularity with engorged vessels and inflammation are evident suggesting trauma or irritation (FIG. 46F). These ranges of histological features indicate the spectrum of the DIGO etiopathological process that is cyclic in nature and can vary from active, vascular and inflamed lesions to well-established, predominantly fibrotic lesions.

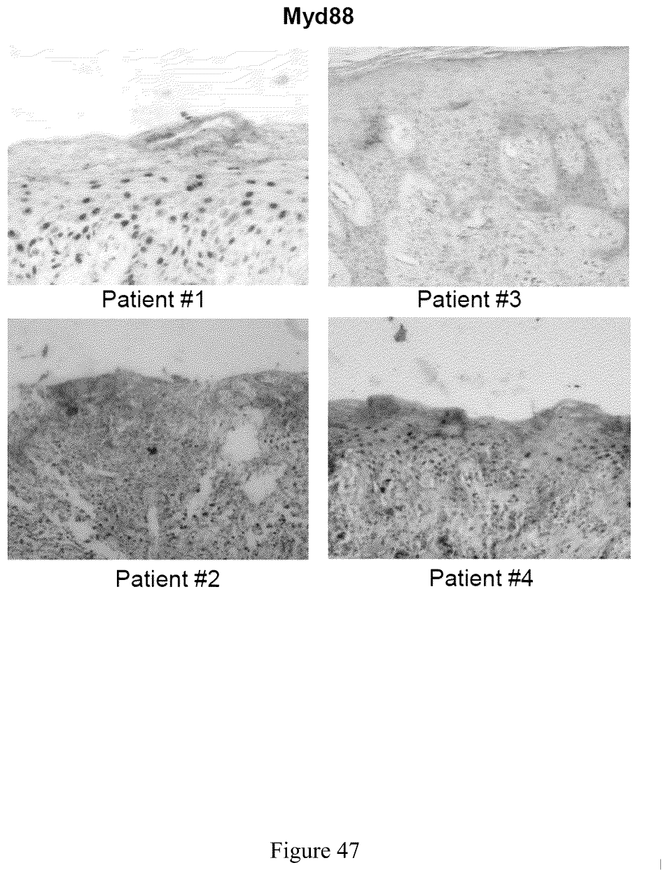

Immunohistochemical analysis. To examine the role of MyD88 in the etiopathological mechanism of DIGO patients, we used immunohistochemistry. Histopathological examination revealed increased epithelial expression of Myd88 (FIG. 47). Quantitative confirmation of these immunostained tissue sections is currently underway.

Drug-induced gingival overgrowth is an unfortunate side-effect of common drugs used in current medical management. Besides the esthetic and functional implications, the fibrotic sequelae in oral gingival tissues are rather unique given its prominent anti-fibrotic nature. Conventional animal models of placing ligatures around the maxillary second molar have major technical challenges. Based on our analysis in this study, we found that an effective site for suture placement to mimic DIGO is the lower, anterior incisors that represent routine human clinical presentation. Suture placements (inducing plaque accumulation) and cyclosporine (drug) injections effectively mimicked the clinical course of DIGO. Our approach provides a technically simple, reliable and reproducible DIGO mice model. In this mouse model, 14 days was adequate to induce the disease and enabled us to test the efficiency of the folic acid in reversing these overgrowths. This is in contrast to the 28 days necessary in the prior described molar ligature model. In this study, we also noted that gingival inflammation and overgrowth could be reduced to some degree, but not completely, by simply removing gingival sutures without antibiotic use as suggested in the prior work. We believe the easy accessibility and relatively rapid time course will make this model very useful to investigate DIGO as well as other gingival inflammation and fibrotic disorders.

Given the curved and complex geometry of gingiva that is further compounded by the disease-induced swellings, routine 2D planimetry is inadequate to accurately estimate gingival overgrowths. The use of stereomicroscopy and 3D digital imaging for volumetric assessment was examined in this study. Stereomicroscopy has been previously used to examine the gingival overgrowth severity as well as a calculation of the ratio of the width (μm) of the second molar to the width of the buccal gingiva (μm). While this technique was found to be visually helpful, it has limited resolution in multiple planes and was noted to be inconsistent for our quantitative analyses. Hence, we investigated 3D planimetry with a digital scanner and digital volumetric reconstruction. We noted this technique provides a high resolution, reliable estimation of the gingival contours. We believe this is the first use of this technique for soft tissue volumetric analyses and will be another valuable tool for future clinical and research studies.

In this study, we were unable to retrieve human DIGO samples despite reaching out to several institutes in the United States. Furthermore, the diagnosis of DIGO is routinely based on medical history and clinical examination with correlation to known drug intake. Hence, the surgical specimens are discarded and rarely submitted for histopathological analysis. Therefore, in this study, we procured de-identified patient archived samples from institutes in India. Our histopathological examinations of H & E-stained DIGO human samples showed pseudoepitheliomatous hyperplasia with elongated rete-ridges, inflammatory infiltration, and fibrous stroma. The histopathological findings of this study were consistent with previously reported studies, which demonstrated epithelial hyperplasia, elongation in the rete ridges, dense and loose connective tissues and inflammatory infiltration. The immunostaining analyses clearly demonstrated increased MyD88 expression in the epithelium indicating a key role for this molecule in the etiopathogenesis of DIGO. Validation in the transgenic mouse model that lacked Myd88 clearly demonstrated its critical role in promoting the fibrotic sequelae of this disease. It is interesting to note that the male mice demonstrated a more aggravated disease phenotype compared to female mice.

A major novelty of this study evaluated the therapeutic benefits of the topical and systemic folic acid supplementation to either prevent or actively treat cyclosporine-induced gingival overgrowth. Folic acid was supplemented in the drinking water (10 μg/ml ad libitum) and methylcellulose gel with the folic acid (100 μg/ml) PLGA microspheres were applied daily on the anterior gingiva. This is in contrast to the previous studies which used only systematic folic acid supplementation or folic acid in a mouthwash. We encapsulated the folic in polylactic-coglycolide (PLG) microspheres and embedded these within methylcellulose gel to enable oral applications. This ensured a longer topical contact time than all prior studies. In our study, the folic acid was very effective in the prevention of the cyclosporine-induced gingival overgrowth. This finding is consistent with the prior studies that reported significant benefits of topical folic acid in prevention of DIGO. Furthermore, we were also able to establish the ability of folic acid treatments in actively reducing previously established gingival overgrowths.

We were able to identify MyD88 gene as a key molecule in the pathogenesis as well as a therapeutic target for folic acid treatments in vivo. Identification of this pathway involved several in vitro studies using cellular, biochemical and molecular analyses. The significant lab analysis aided us in designing precise dosimetry and a molecular targeted therapy for folic acid in DIGO. The mechanistic role of the epithelium leading the fibrotic response has not speculated in DIGO previously. However, we believe this is the first demonstration of a causal pathway in the disease process. Prior work has predominantly focused on the role of collagen production and fibroblast proliferation. Several studies have also examined the role of the inflammatory cells in mediating the ECM synthesis and turnover. The histopathological analyses of both mice and human tissues clearly revealed epithelial hyperplasia and this reactive feature suggested it may have a role in the disease process leading to our exploration.

Conclusions and implications of this study: A novel DIGO mouse model was developed in this study. We found the anterior gingiva effective site for suture placement to mimic routine human clinical presentation. Moreover, suture placement and cyclosporine injection provide a technically simple, reliable and reproducible DIGO mice model. Analyses from mice and human DIGO tissues revealed a key role for Myd88 in mediating DIGO. Further, results from this study indicate folic acid is effective at preventing and treating cyclosporine-induced gingival overgrowth. Further, it appears that folic acid mediates its therapeutic benefits by downregulating Myd88 in DIGO.

### EXAMPLE 3

This example provides results of using compositions of the present disclosure.

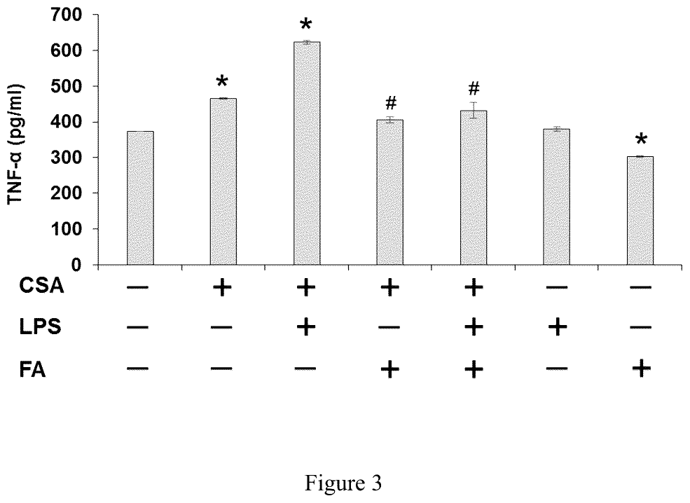

Folic acid reduces TNF-α oral keratinocytes. Oral Keratinocytes were seeded in 60 mm plates with a density of 2.5×10⁴/cm². After adhesion, the cells were treated with different concentration of folic acid (2.5 μg/ml-40 μg/ml) and amount of TNF-α was measured by ELISA (p<0.05). See FIG. 2.

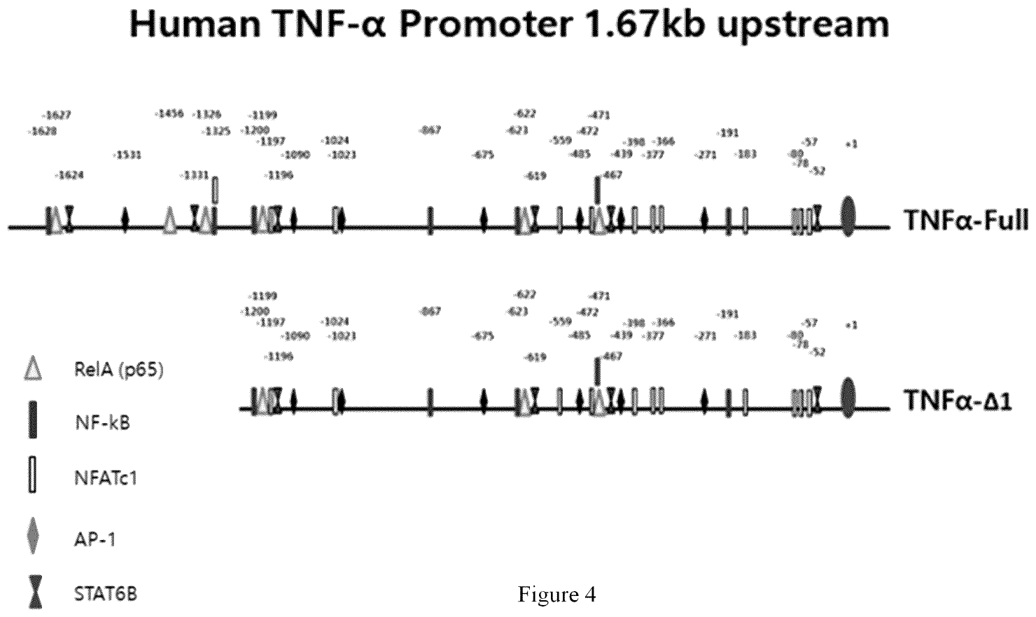

Folic acid reduces TNF-α in Oral Keratinocytes on LPS stimulation. Oral Keratinocytes were seeded in 60 mm plates with a density of 2.5×10⁴/cm². After adhesion, the cells were treated with LPS (10 μg/ml), CSA (10 μg/ml), and FA (10 μg/ml) for 24 hours. The amount of secreted TNF-α was determined by ELISA (p<0.05). See FIG. 3.

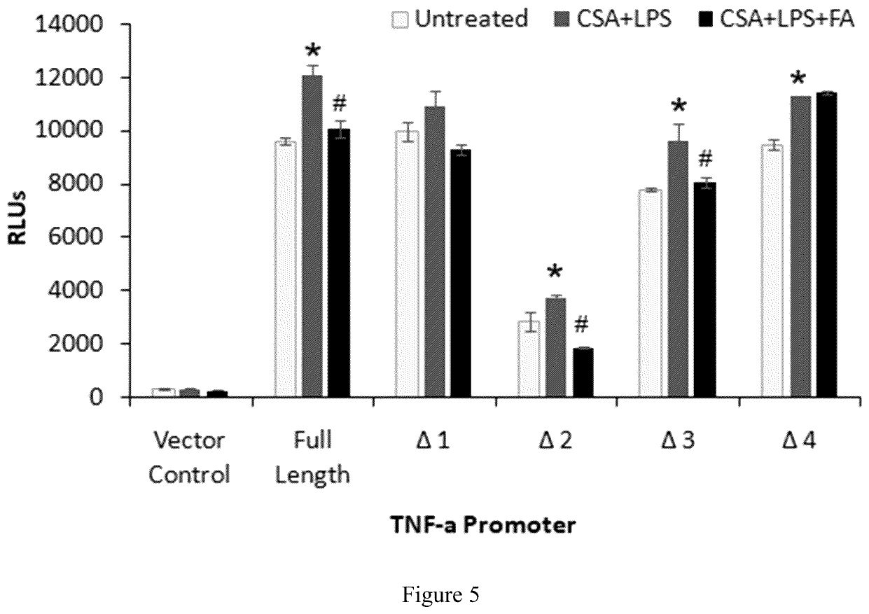

Folic acid inhibits promoter activity of TNF-α in Oral Keratinocytes cells. Oral Keratinocytes cells were transfected with Full length TNF-α promoter and four deletion constructs (Δ1-4) and treated with CSA (10 μg/ml) & LPS (10 μg/ml), and CSA (10 μg/ml), LPS (10 μg/ml) & Folic acid (10 μg/ml). TNF-α luciferase reporter activity were measured. (p<0.05). See FIG. 5.

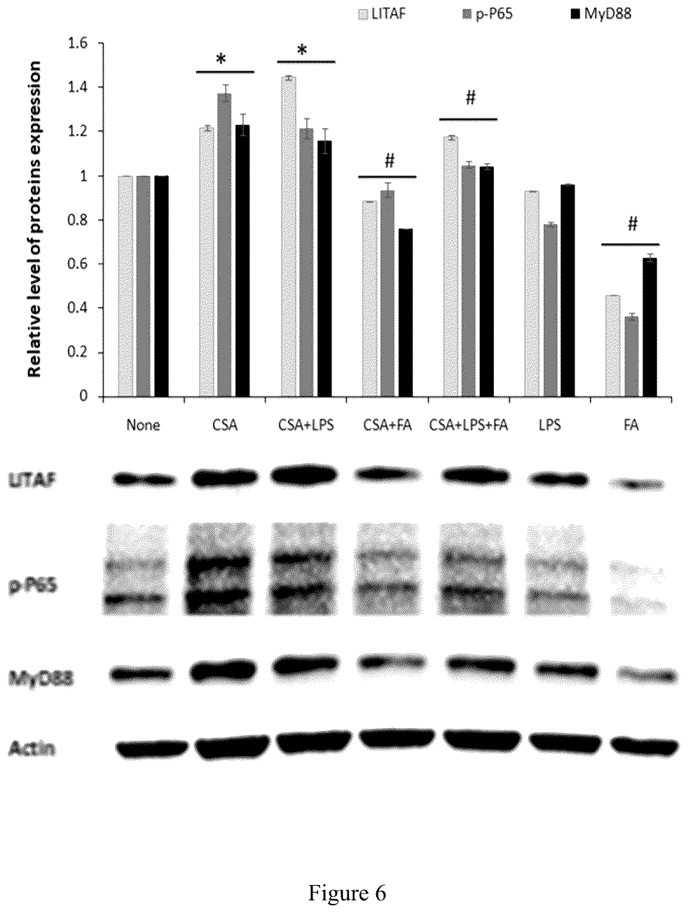

Folic acid inhibits CSA-induced expression of LITAF, p-P65, and MyD88 proteins in Oral Keratinocytes cells oral Keratinocytes were seeded in 60 mm plates with a density of 2.5×10⁴/cm² and treated with LPS (10 μg/ml), CSA (10 μg/ml), and FA (10 μg/ml) for 24 hours and determined the LITAF, MyD88, and p-p65 proteins level. Oral Keratinocytes cells were cultured, treated with CSA, LPS or FA and incubated for 24 hours. See FIG. 6.

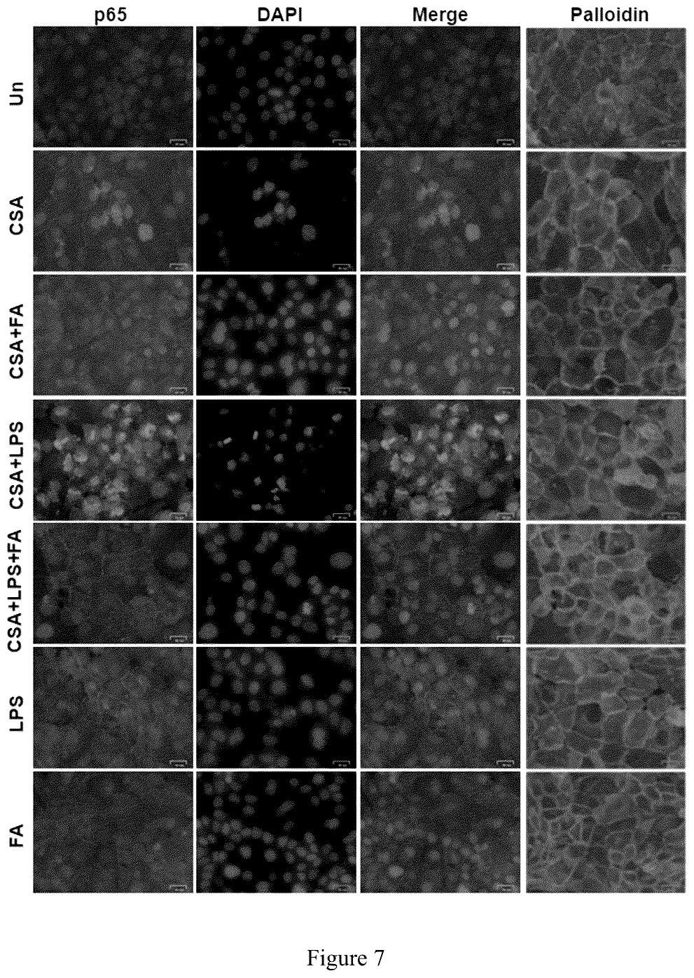

Folic acid inhibits CSA-induced activation of phospho-P65 in Oral Keratinocytes cells. Oral Keratinocytes cells were cultured, and treated with CSA, LPS or FA alone or with combinations and incubated for 24 hours. The immunofluorescent images were taken to determine the translocation of p-P65 (green) to the nucleus (blue). Cells were counterstained with Phalloidin (cytoskeleton, red) and DAPI (nucleus). See FIG. 7.

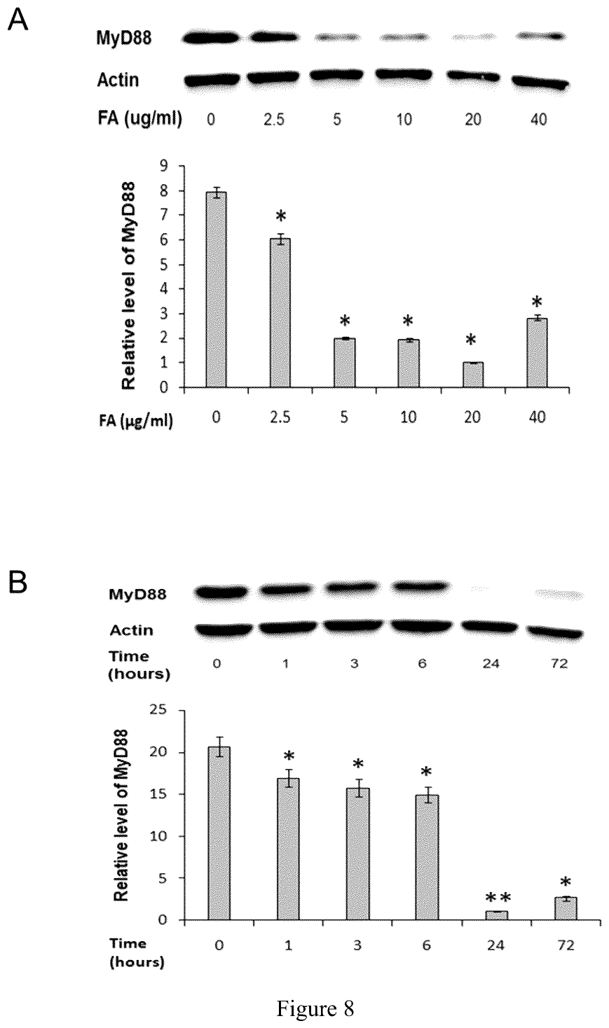

Folic acid inhibits oral keratinocyte cells. Oral Keratinocytes cells were cultured, and treated with FA with different concentration, and different time point with a concentration of 10 μg/ml and determined the Dose-dependent effects of FA on MyD88 assessed with western blots analysis at 24 hrs. Kinetics of MyD88 downregulation following FA treatments assessed with western blots. See FIG. 8.

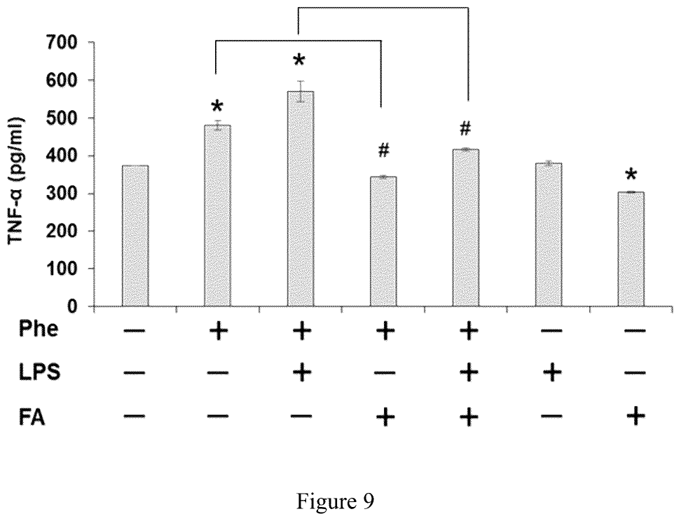

Folic acid inhibits Phenytoin-induced TNF-α in Oral Keratinocytes cells. Oral Keratinocytes were seeded and treated with LPS (10 μg/ml), Phe (10 μg/ml), and FA (10 μg/ml) for 24 hours. The amount of secreted TNF-α was determined by ELISA. (p<0.05). See FIG. 9.

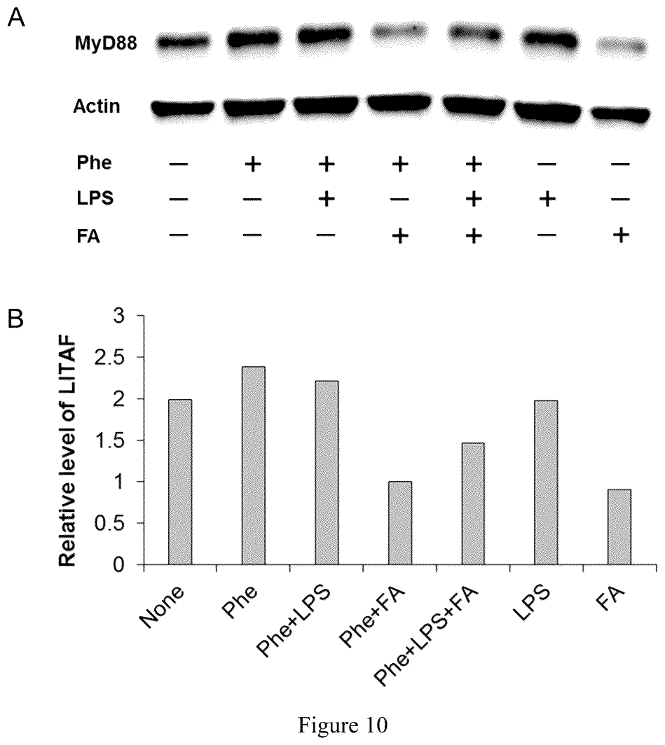

MyD88 expression is reduced by folic acid treatments. Oral Keratinocytes were seeded in 60 mm plates. The cells were treated with LPS (10 μg/ml), Phe (10 μg/ml), and FA (10 μg/ml) for 24 hours. The amount of MYD88 protein was determined by Western blot analysis. The protein bands were analyzed quantitatively by using ImageJ software. See FIG. 10.

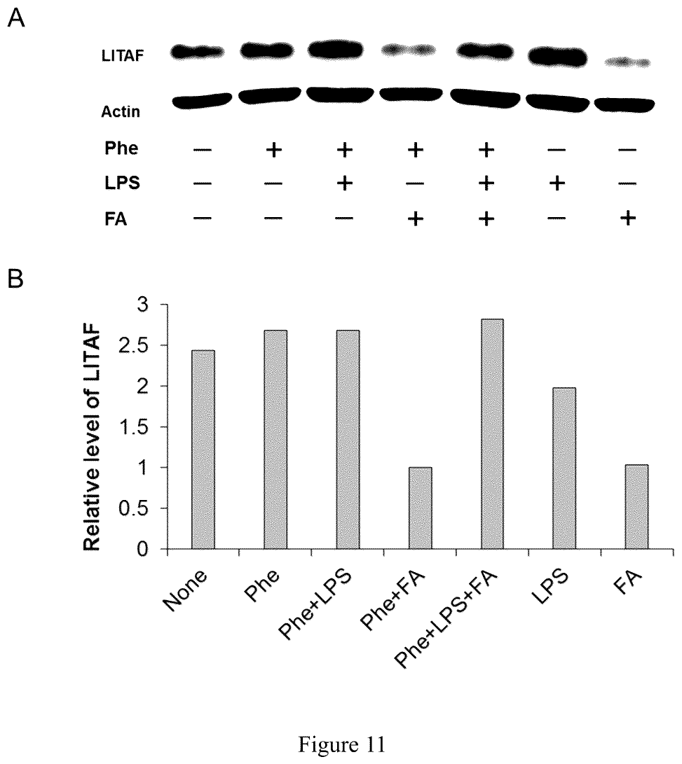

LITAF expression is reduced by folic acid following drug treatments. Oral Keratinocytes were seeded in the plates. The cells were treated with LPS (10 μg/ml), Phe (10 μg/ml), and FA (10 μg/ml) for 24 hours. The amount of LITAF protein was determined by Western blot analysis. The protein bands were analyzed quantitatively by using ImageJ software. See FIG. 11.

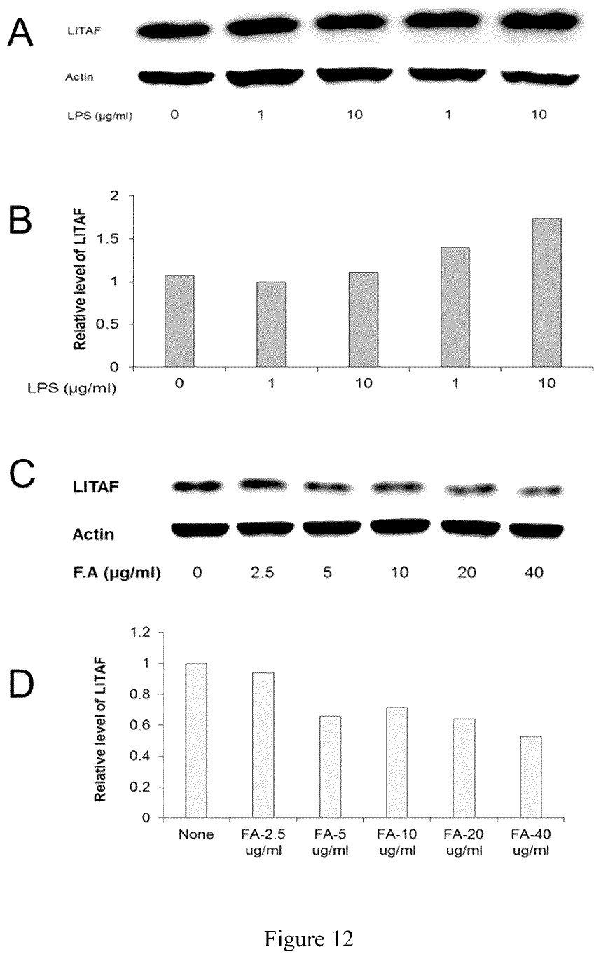

Oral Keratinocytes cells were treated with different serotypes (026:B6, and 055:B5) and concentrations (1 μg/ml & 10 μg/ml) of LPS, and different concentrations of folic acid (2.5-40 μg/ml) for 24 hours and measured the amount of LITAF protein by Western blot analysis. The protein bands were analyzed quantitatively by using ImageJ software. See FIG. 12.

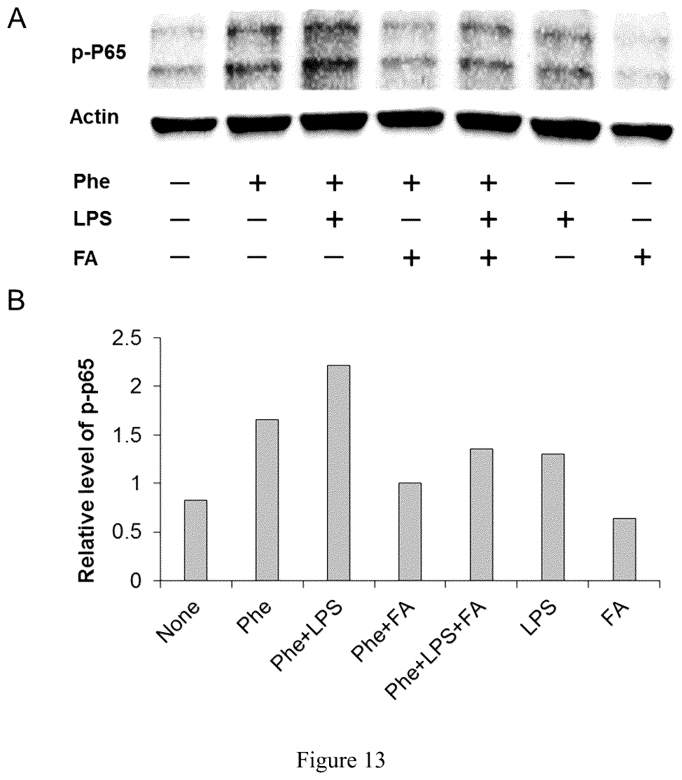

Folic acid reduces phosphorylation of NF-κB (p-P65). Oral Keratinocytes cell were seeded in the plates. The cells were treated with LPS (10 μg/ml), Phe (10 μg/ml), and FA (10 μg/ml) for 24 hours. The amount of phospho-p65 (p-P65) protein was determined by Western blot analysis. The protein bands were analyzed quantitatively by using ImageJ software. See FIG. 13.

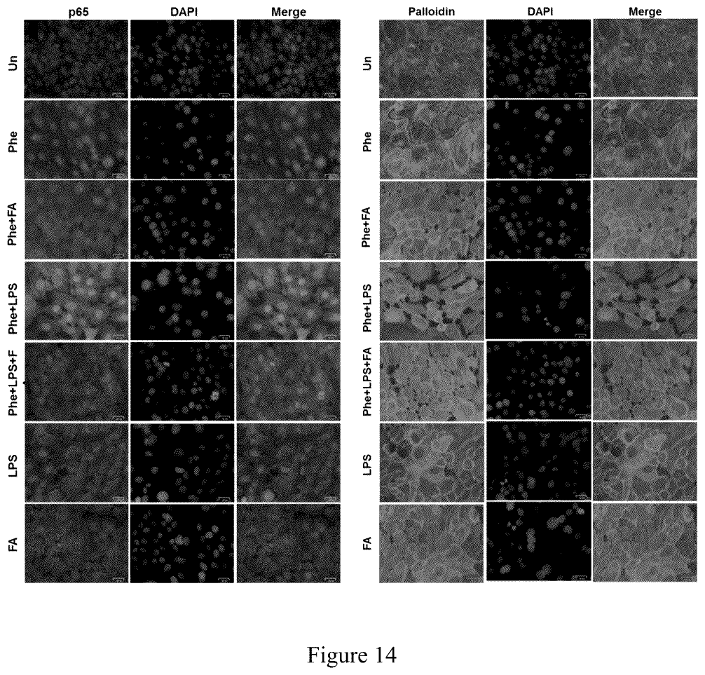

Folic acid reduced nuclear translocation of p-P65. Oral Keratinocytes cells were seeded in 24 well plates. The cells were treated with LPS (10 μg/ml), Phe (10 μg/ml), and FA (10 μg/ml) for 24 hours. Translocation of phospho-p65 (p-P65) protein to the nucleus was determined by fluorescence microscopy as shown in the green color. The morphology of cells and nucleus position were determined by palloidin and DAPI staining shown by red and blue colors respectively. See FIG. 14.

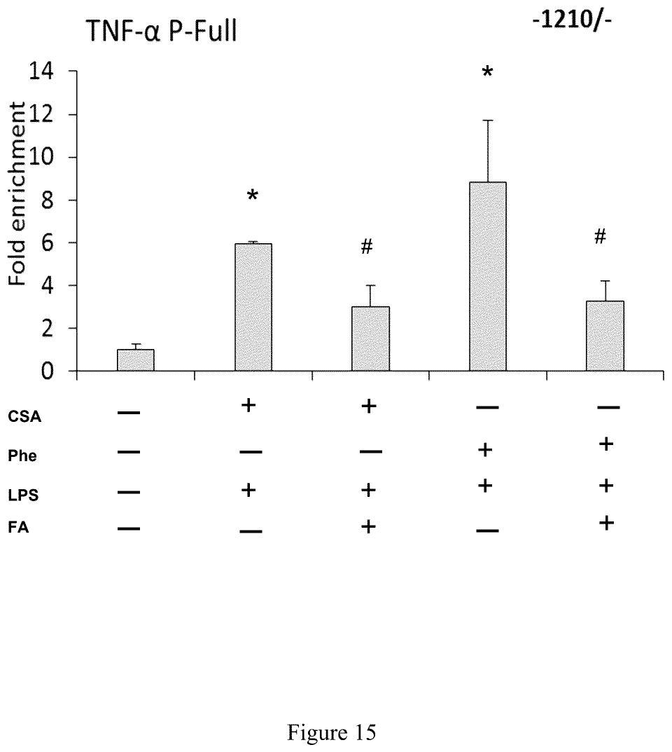

A ChIP-qPCR assay demonstrated that LITAF binds to the TNF-α promoter. Oral Keratinocytes cells were seeded in 24 well plates. The cells were treated with LPS (10 μg/ml), Phe (10 μg/ml), LPS (10 μg/ml), and (FA (10 μg/ml) for 24 hours. CHIP-qPCR assay was performed to determine the binding of LITAF to the Human TNF-α promoter. *p<0.05 (treated vs non-treated), #p<0.05 (with vs without FA). See FIG. 15.

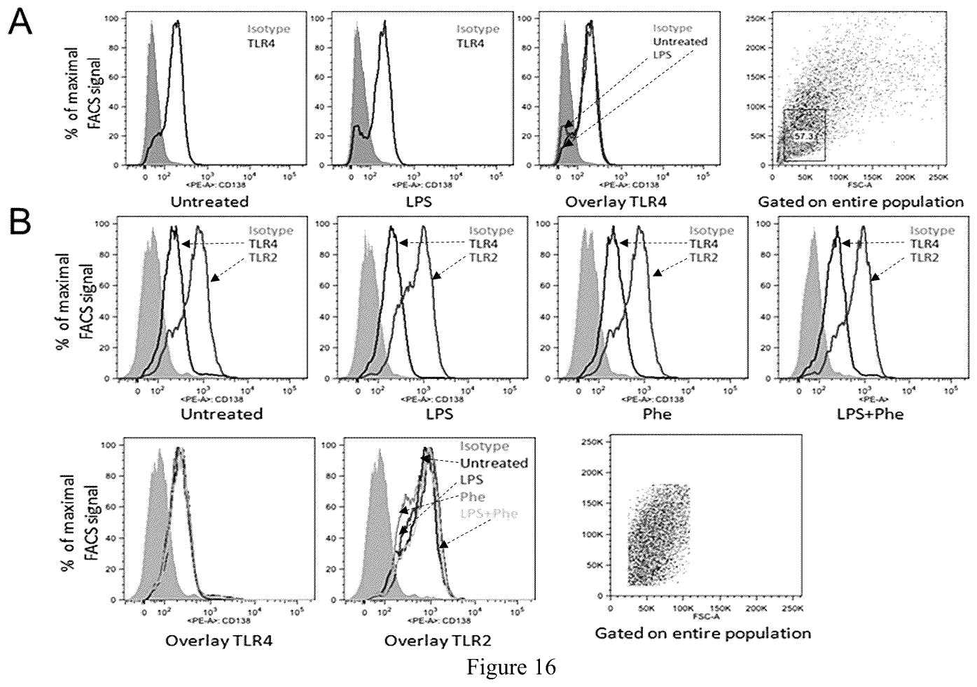

Folic acid treatments do not alter TLR2 and TLR4 expression. Oral Keratinocytes Cells were treated with only LPS (10 μg/ml) for 24 hours. The FACS assay analysis shows the amount of TLR4 receptor on the surface of Oral Keratinocytes cells. Cells were treated with LPS (10 μg/ml) and Phe (10 μg/ml) for 24 hours. The FACS assay analysis shows the amount of TLR2 and TLR4 receptor on the cells surface. Cells were treated with LPS (10 μg/ml), Phe (10 μg/ml), and FA (5, 10, and 10 μg/ml) for 24 hours. The FACS assay analysis shows the amount of TLR2 and TLR4 receptors on the surface of Oral Keratinocytes cells. FACS assay analysis shows the TLR2 and TLR4 receptors on the surface of Oral Keratinocytes cells after treatment with LPS, Phe, and different concentration of folic acid. FIG. 16.

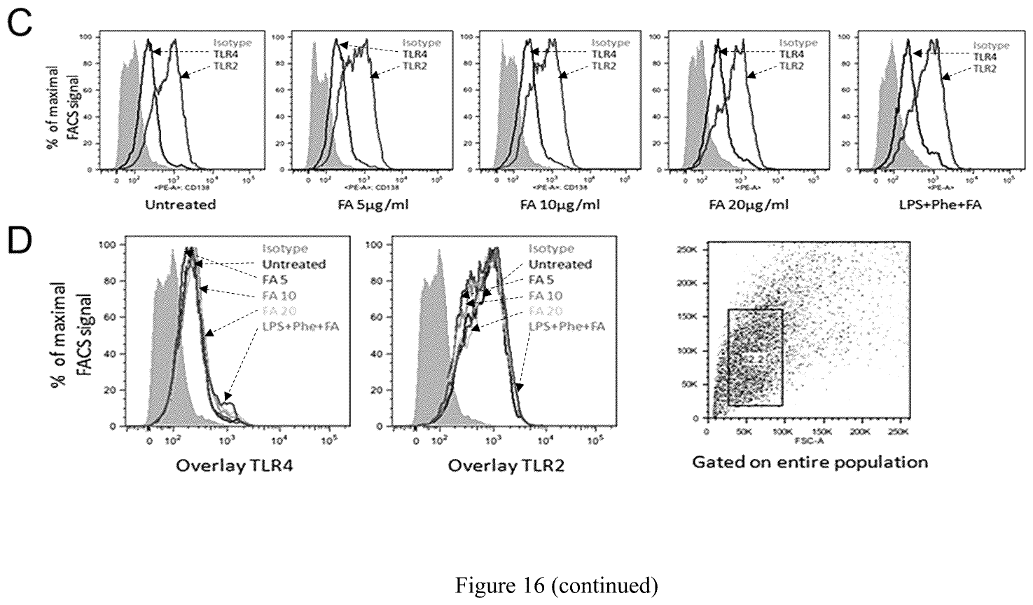

Folic acid treatments reduce TGF-β expression in Oral Fibroblast via inhibiting TNF-α signaling in Oral Keratinocytes. Human Oral Fibroblast (HOF) cells were cultured and treated with CSA, Phe, LPS, TGF-β inhibitor (SB), TNF-α inhibitor, rhTNF-α (TNF-α protein), and conditioned media (CM) collected from Oral Keratinocytes cells. ELISA results shows the amount of TGF-β secreted by HOF cells. See FIG. 17.

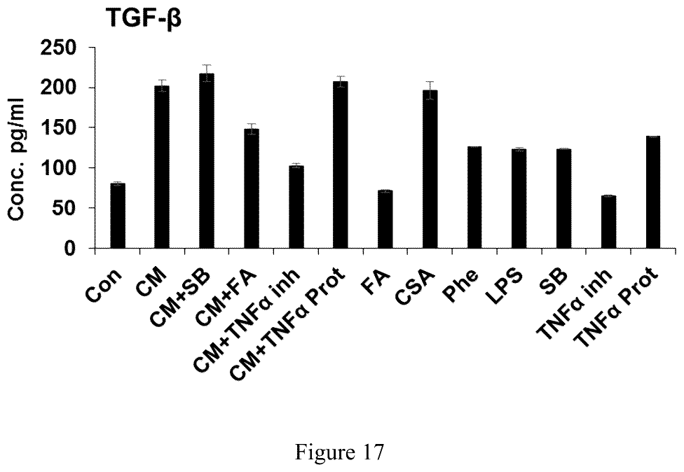

Folic acid reduces the collagen expression through TNF-α-activated TGF-β signaling in Human Oral Fibroblasts. Human Oral Fibroblasts (HOF) cells were seeded in 60 mm plates with a density of 2.5×10⁴/cm² and treated with condition (CM) media collected from Oral Keratinocytes containing LPS (10 μg/ml), CSA (10 μg/ml), Phenytoin (10 μg/ml), or Folic acid (10 μg/ml) and incubated for 24 hours. The Collagen type I alpha 1 (Col1a1) gene expression was determined by QPCR. HOF cells were treated with rTGF-β, SB-431542 (SB), rTNF-α, or TNF-α inhibitor (TNF-α inh). Quantitative real-time PCR results showing the expression of Col1a1 in HOF cells after the treatment. (p<0.05). See FIG. 18.

The in vivo model of DIGO used two different normal (wild type) mice strains: 1. C57BL/6 and 2. BALB/c. Transgenic mice for Myd88 knockout mice and wild type littermates were used. Both male and female mice were used. Ligature was placed around the lower central incisors and mice were injected with cyclosporine A (CsA). The delivery system was folic acid in polymer (PCL/PLGA) microspheres formulated into a gel.

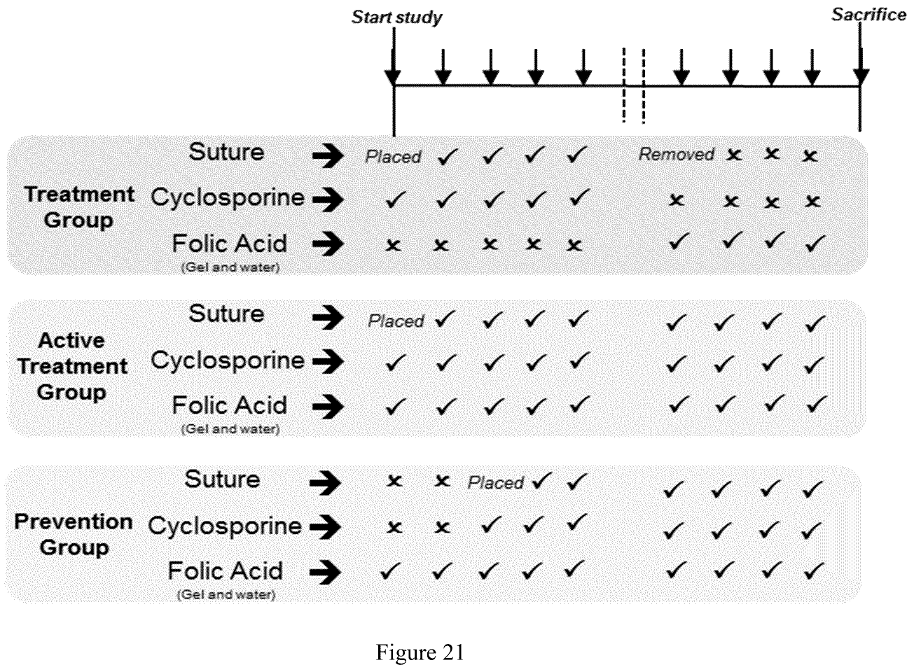

The in vivo DIGO model was used to assess efficacy of folic acid treatments using folic acid-encapsulated PLGA microspheres mixed with methylcellulose gels. This was applied to mice gingiva. Silk sutures were placed around incisors to facilitate plaque accumulation and induce inflammation. See FIG. 22.

The in vivo DIGO models were used to assess efficacy of folic acid treatments. Four weeks male C57/B6 mice were subjected to ligature placement and Cyclosporine (CsA) to induce gingival swelling, the top panel shows the time course of interventions while lower panels shows clinical images of placement and removal of sutures. The Folic acid (FA) treatment group was started at 1 week after establishment of DIGO and sutures were removed and CsA injections were stopped. See FIG. 23.

To assess gingival swelling in the treatment group, the mice were sacrificed. Following sacrifice, mice jaws were dissected and imaged using stereomicroscopy and specimens were photographed with a digital camera. In these studies, mice were divided into two groups where both groups received CsA and suture for 1 week followed by removal of sutures and stopping CsA injections. One group then received Folic acid applications daily (Treatment group) until the end of the study. See FIG. 24.

To assess gingival swell in the prevention group, the mice were sacrificed. Following sacrifice, mice jaws were dissected and imaged using stereomicroscopy. The specimens were photographed with a digital camera on their occlusal, buccal, and lingual surfaces. Mice were divided into three groups namely, untreated (control), Disease (CsA & sutures) and Prevention (CsA, sutures & Folic acid). See FIG. 25.

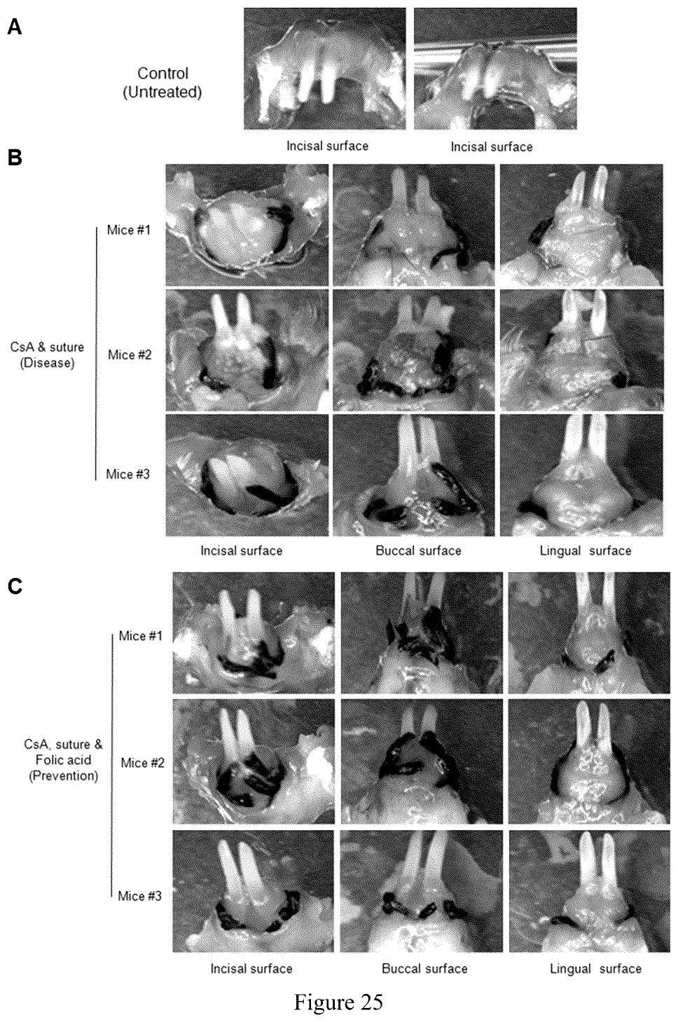

Gingival samples were subjected to western blots to assess signaling pathways involved in DIGO. See FIG. 26.

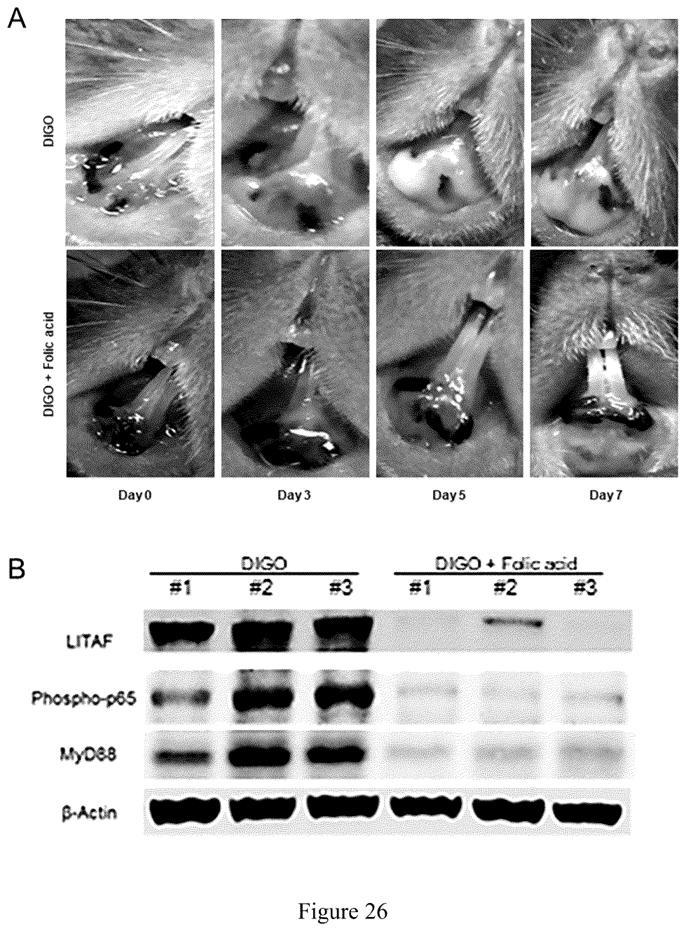

Four weeks-male BALB/c mice had a suture place between the incisors and injected with Cyclosporine (CsA) alone or in combination with folic acid (CsA & FA) for prevention. Digital images were captured over a time course. Digital 3D planimetry was performed to assess gingival enlargements in two mice in both groups at Day 8. See FIG. 27.

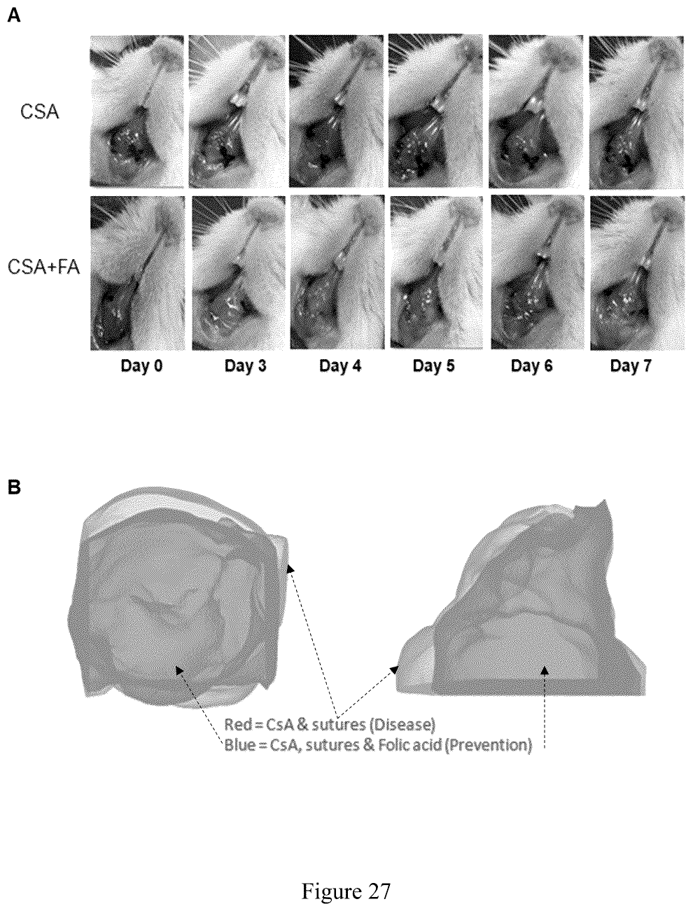

Gingival tissues of the prevention group were evaluated. Mice were sacrificed and total proteins isolated from dissected gingival tissues. Expression of LITAF, Phospho-p65, and MyD88 in gingival tissue lysates were examined by western blot analysis. Band intensity of western blot was normalized to β-actin and quantitated by densitometry. See FIG. 28.

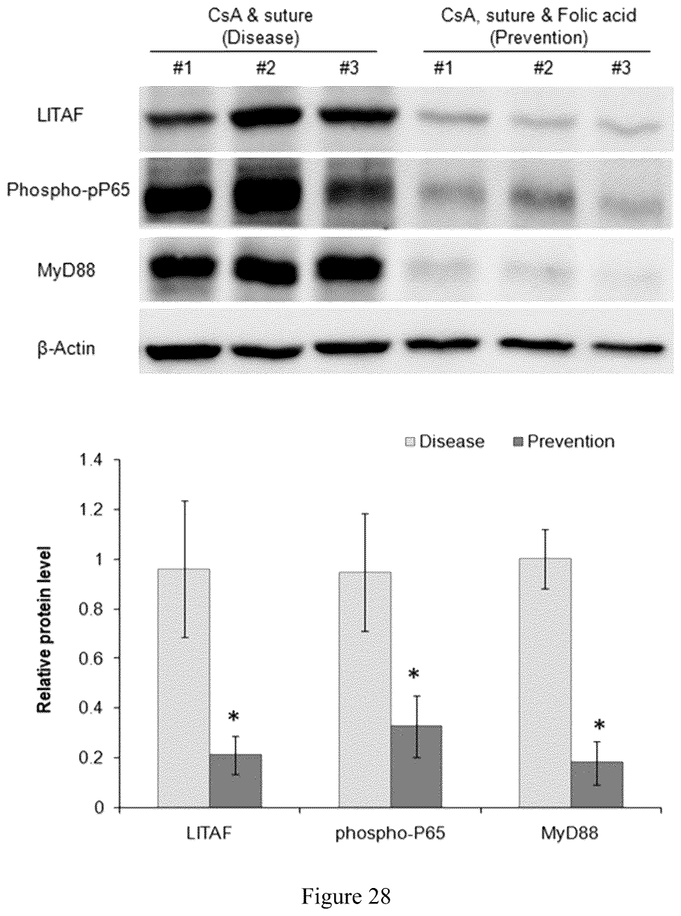

Folic acid reduced the expression of TNF-α in mice gingival tissues. Mice gingival tissue lysates were assessed for TNF-α levels using an ELISA (n=5). See FIG. 29.

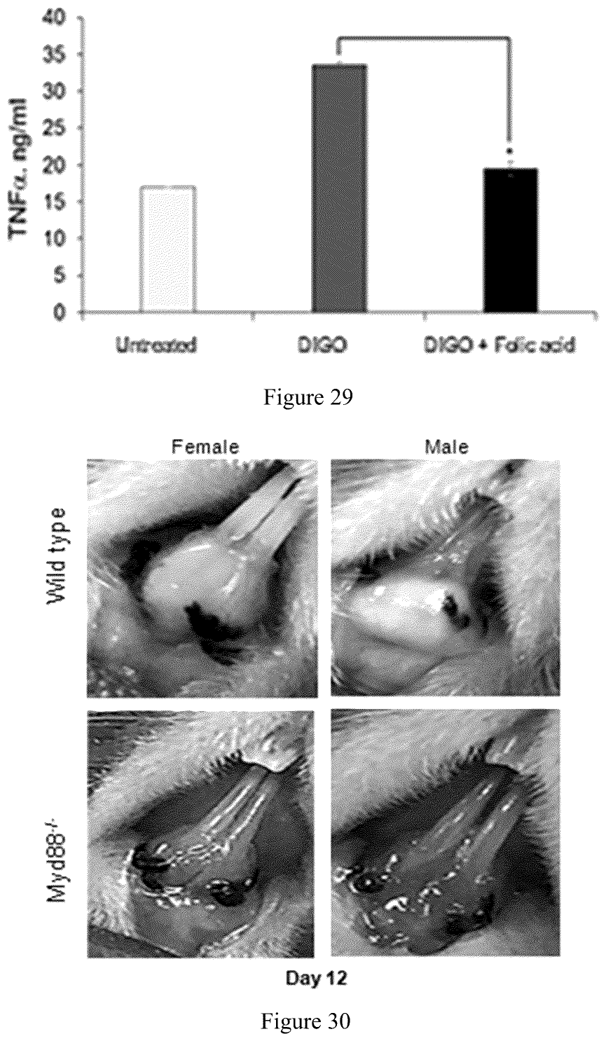

DIGO was induced in male and female mice in wildtype and Myd88^{-/-} (knockout) mice and images of their gingival tissues were captured. See FIG. 30.

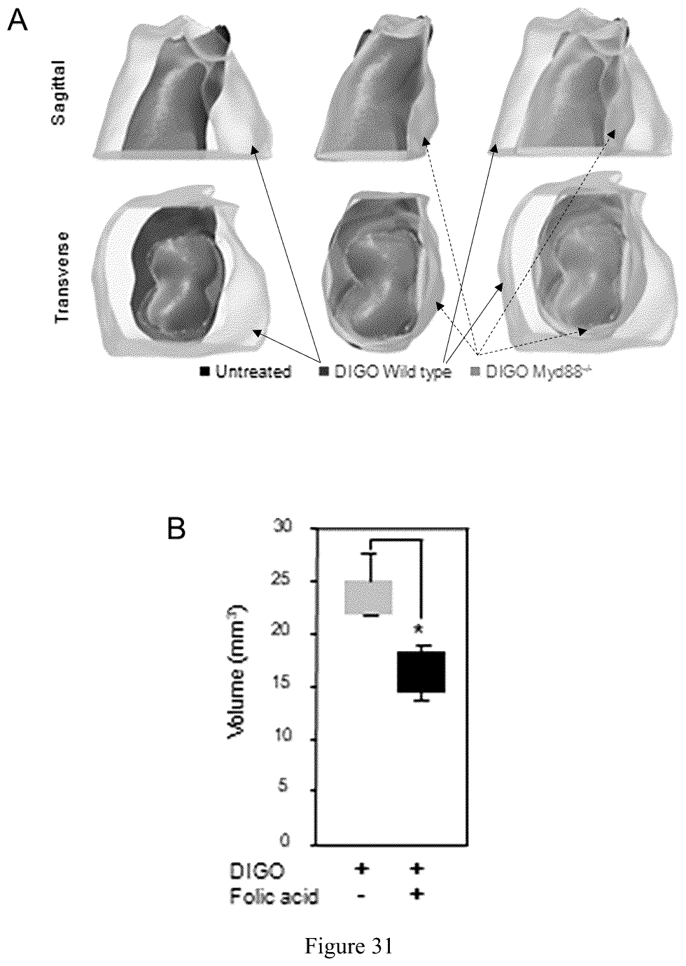

Wild type mice have significant DIGO induction compared to MyD88 knockout mice. Digital 3D planimetry to assess gingival enlargements in surrounding the incisors. Quantification of gingival enlargement in the two groups. See FIG. 31.

Although the present disclosure has been described with respect to one or more particular embodiments and/or examples, it will be understood that other embodiments and/or examples of the present disclosure may be made without departing from the scope of the present disclosure.

1. A composition comprising a polymeric gel comprising a plurality of microspheres, wherein the individual microspheres of the plurality of microspheres encapsulate folic acid.

2. The composition of claim 1, wherein the polymeric gel is chosen from a methylcellulose gel, chitosan gel, polylactic glycolic acid gel, and combinations thereof.

3. The composition of claim 1, wherein the polymeric gel is methylcellulose gel.

4. The composition of claim 3, wherein the polymer gel comprises phosphate buffered saline and 1-5% weight by volume methylcellulose.

5. The composition of claim 1, wherein the plurality of microspheres are chosen from polylactic glycolic acid microspheres, polycaprolactone (PCL) microspheres, chitosan microspheres, starch microspheres, polylactic acid microspheres, poly(ester amide) microspheres, alginate microspheres, and combinations thereof.

6. The composition of claim 5, wherein the microspheres are polylactic glycolic acid microspheres.

7. The composition of claim 1, wherein the concentration of folic acid is 2.5 to 150 μg/mL.

8. The composition of claim 1, wherein the concentration of microspheres is 0.001 to 0.07 mg/μL.

9. The composition of claim 1, wherein the plurality of microsphere(s) further comprise one or more drugs, one or more peptides, one or more growth factors, one or more nucleic acids, or combinations thereof.

10. The composition of claim 9, wherein the one or more drugs are chosen from antibiotics, anti-inflammatoires, antibodies and combinations thereof.

11. A method for treating an individual afflicted with or suspected of having drug-induced gingival overgrowth (DIGO) comprising:
(a) obtaining a scan, mold, or model of the individual's oral cavity where a device will fit;
(b) fabricating the device corresponding to the individual's scan, mold, or model of the oral cavity with a polymer;
(c) generating the composition of claim 1;
(d) coating the device with the composition;
(e) administering the completed device to the individual for sufficient time to treat the DIGO; and
(f) optionally, monitoring the individual's response to the device treatment.

12. The method of claim 11, wherein the device is chosen from a splint, mouth guard, full denture, partial denture, and filling.

13. The method of claim 11, wherein the polymer is chosen from polymethylmethacrylate (PMMA) polymers, bis-GMA polymers, polyester polymers, polyacrylate polymers, polyvinyl polymers, epoxy polymers, and combinations thereof.

14. The method of claim 11, wherein the polymeric gel is a methylcellulose gel.

15. The method of claim 11, wherein the plurality of microspheres are polylactic glycolic acid microspheres.

16. The method of claim 11, wherein the plurality of microsphere(s) further comprise one or more drugs, one or more peptides, one or more growth factors, one or more nucleic acids, or combinations thereof.

17. A method for treating an individual afflicted with or suspected of having drug-induced gingival overgrowth (DIGO) comprising administering to the individual the composition of claim 1 for a sufficient time to treat the DIGO.

18. The method of claim 17, wherein the polymeric gel is a methylcellulose gel.

19. The method of claim 17, wherein the plurality of microspheres are polylactic glycolic acid microspheres.

20. The method of claim 17, wherein the plurality of microsphere(s) further comprise one or more drugs, one or more peptides, one or more growth factors, one or more nucleic acids, or combinations thereof.
